(19) United States
(12) Patent Application Publication
(10) Pub. No.: US 2008/0145346 A1
(43) Pub. Date: Jun. 19, 2008

(54) Methods And Compounds For Altering The Load Of Hepatitis Virus

(75) Inventors: Fong Poh Lisa Ng, Singapore (SG); Ee Chee Ren, Singapore (SG)

Correspondence Address: FOLEY & LARDNER LLP, P.O. BOX 80278, SAN DIEGO, CA, 92138-0278, US

(73) Assignee: Agency for Science, Technology and Research, Singapore (SG)

(21) Appl. No.: 10/583,068

(22) PCT Filed: Nov. 12, 2004
(86) PCT No.: PCT/SG04/00368

§ 371 Date: May 4, 2007

Related U.S. Application Data
(60) Provisional application No. 60/529,632, filed on Dec. 16, 2003.

(51) Int. Cl. A61K 48/00; C12Q 1/70; C12Q 1/68

(52) U.S. Cl. 424/93.21; 514/44; 435/5; 514/12; 435/6

(57) ABSTRACT

The present invention relates to a method for altering the load of a Hepatitis virus present in an infected host organism. The method involves modulation of the complex formation of a heterogeneous nuclear ribonucleoprotein K (hnRNP K) and the regulatory region of a Hepatitis virus, enhancer II region. Additionally there are methods of identifying compounds that modulate complex formation, and the use of such compounds in diagnosis of a Hepatitis infection. The present invention also relates to a mutation in enhancer II region at position 1752 of the virus sequence which reduces the binding affinity of hnRNP K with the enhancer II region.

The present invention relates to a method for altering the load of a Hepatitis virus present in a host organism that is infected with such virus. This method comprises the modulation of the complex formation of a heterogeneous nuclear ribonucleoprotein (hnRNP) K or a functional fragment thereof with a regulatory region on the Hepatitis virus genome. Additionally, the invention relates to methods of identifying compounds that are able to modulate said complex formation. The present invention also relates to compounds that are able to achieve such modulation, such as nucleic acid molecules, immunoglobulins, antagonists and agonists of cell surface receptors, compounds that modulate the degree of phosphorylation of hnRNP K proteins, as well as compounds that modulate the intracellular quantity of hnRNP K proteins. Finally, the invention relates to the use of such compounds for the diagnosis of Hepatitis infection.

Hepatitis B and Hepatitis C viruses are two of seven known viruses (Hepatitis A, B, C, D, E, G, and TT viruses) that together account for the majority of cases of viral Hepatitis. Hepatitis caused by infection with Hepatitis B or C virus is a major worldwide health problem and one of the most common infections in the world today. More than 400 million people worldwide are chronically infected by the Hepatitis B Virus (HBV), more than 170 million people by the Hepatitis C virus (HCV) (for respective reviews see Ching L L et al., Lancet 362(9401), 2003, 2089-2094; Poynard, T et al., Lancet 362(9401), 2003, 2095-2100). As a comparison, the number of people infected with human immunodeficiency virus (HIV) is estimated to be 60 million. Chronic Hepatitis is associated with serious complications such as: liver failure; chronic Hepatitis progressing to cirrhosis; cirrhosis with active Hepatitis at risk for variceal bleeding, ascites, and hepatocellular carcinoma; chronic Hepatitis with high viremia at risk for transmitting Hepatitis virus; chronic Hepatitis with extrahepatic complications. Hepatitis C is the major cause of liver transplantation in Europe and the USA. Hepatitis A does not cause chronic disease, however evidence suggests that acute Hepatitis A superimposed on chronic liver disease can result in a more severe disease and higher fatality rate (Cooksley G, J Gastroenterol Hepatol. 19 (Suppl 1), 2004, S17-20). The Hepatitis D virus is an incomplete virus that has HBV infection as a prerequisite. Superinfection with Hepatitis D often causes progression from acute Hepatitis to liver cirrhosis (Bean P, Am Clin Lab. 21 (5), 2002, 25-27). While the Hepatitis E virus can cause an acute, self-limited, icteric hepatitis (Wang L, Zhuang H, World J Gastroenterol. 10 (15), 2004, 2157-2162), Hepatitis G virus infection has not been found to be associated with any known disease state (Stapleton J T, Semin Liver Dis. 23 (2), 2003, 137-148), although it is common and frequently persists in humans. The impact of TT virus on liver diseases is currently uncertain (Hino S, Rev Med. Virol. 12 (3), 2002, 151-158).

Although treatments for HBV or HCV are available, none has so far resulted in a complete eradication of the virus in chronically infected patients. The commonly desired endpoint of treatment of HBV or HCV today is therefore limited to the development of immunoglobulins, i.e. antibodies, or small molecules, and a stable suppression of virus replication to levels that are associated with disappearance of intrahepatic necrosis and inflammation and slowing down of the fibrosis progression.

Of the drugs currently available for the treatment of HBV and HCV, interferon α is known to be suitable for the treatment of both viruses. Interferon α is a molecule that is produced by cells of the immune system; it is secreted in response to viruses and other invading agents. Interferon α has suboptimal pharmacokinetics leading to significant fluctuations in blood levels (Reddy K R et al., Advanced Drug Delivery Reviews 54 (4), 2002, 571-586; Ferenci P, it J Clin Pract. 57 (7), 2003, 610-615), which resulted in the application of “PEGylated interferon”, which is created by attaching a polyethylene glycol molecule, such as a large, branched, 40-kD polymer.

Two additional drugs are currently available for treatment of HBV, Lamivudine and Adefovir; both of them are nucleoside analogues. As such, they block viral amplification by inhibiting the viral enzyme nucleoside reverse transcriptase. As side effects, they can cause serious damage to the liver and lactic acidosis (for a general overview see Roche B, Samuel D, Liver Transpl. 10 Suppl, 2004, S74), renal toxic side effects have also been reported. A major drawback is the occurrence of resistant variants of HBV containing a mutation in nucleoside reverse transcriptase. Both drugs are generally used in combination with interferon (Marcellin P et al., N Engl J Med 351 (12), 2004, 1206-1217). Other compounds based on a similar mechanism of action have gone through phase 2 trials.

For treatment of HCV there is currently only one drug, called Ribavirin, in addition to interferon α available, also being a nucleoside analogue. It is generally used in combination with interferon as well. The precise mechanism of its therapeutic action is still not fully understood. It is generally thought to inhibit viral RNA synthesis indirectly through a decrease in levels of intracellular guanosine triphosphate (GTP) by inhibiting a certain enzyme. Its effect on HCV replication is however small (Reichard O et al., Lancet 351 (9096), 1998, 83-87). Furthermore, hematologic abnormalities such as anemia, neutropenia, and thrombocytopenia are common side effects (Ong J P, Younossi Z M, Cleve Clin J. Med. 71, Suppl 3, 2004, S17-21).

While a therapy using a combination of existing drugs appears to be more effective in the treatment of HCV infection (Poynard et al., supra; Reichard O et al, supra), for HBV infection such additional benefits have so far not even convincingly been shown (Ching L L et al., supra). Furthermore in any of the above therapies most patients do not achieve long-term responses (Marcellin P et al., supra; Papatheodoridis G V, Hadziyannis S J, Aliment Pharmacol Ther. 19 (1), 2004, 25-37). As an example, studies showed that sustained responses to interferon treatment were found in only 10-20% of patients with HCV infection (Reichard O et al, supra). Therefore there is a need for the search for alternative targets and methods of treating Hepatitis infection.

Such a search is preferably based on a detailed understanding of the build-up and function of the corresponding viruses. While for HBV some detailed information on the elements underlying the control of its replication is available, little is for example so far known about HCV. HBV is a DNA virus of the hepadnavirus family, which replicates via an RNA intermediate. The genome of Hepatitis B Virus is a 3.2 kilobase circular partially double-stranded DNA containing four overlapping genes, three promoter and two enhancer regions. Either of the two differentially regulated enhancers is able to enhance activity of all three promoters (Su H, Yee J K, Proc Natl Acad Sci USA 89 (7), 1992, 2708-2712).

HCV is a RNA virus of the flavivirus family. Its single-stranded 10 kilobase genome contains a single gene. Further investigations of this genome have been hampered by the fact that HCV from a single isolate cannot be defined by a single sequence but rather by a population of variant sequences closely related to each other. This diversity corresponds to genetically distinct groups or genotypes (Martell et al., Nucleic Acids Research 32 (11), 2004, 90). There are indications that the 3′ nontranslated region contains both signals that are essential for replication as well as regulatory regions (Yi M, Lemon S M, J Virol. 77 (6), 2003, 3557-3568).

The Hepatitis D virus is an incomplete virus (Bean, supra), and the Hepatitis E virus (HEV) is an unclassified, small, single-stranded non-enveloped RNA virus with a 7.2 kilobase genome (Wang & Zhuang, supra). The Hepatitis G virus is an enveloped RNA virus belonging to the Flaviviridae family (Halasz R et al., Scand J Infect Dis. 33 (8), 2001, 572-580). The TT viruses are apparently heterogeneous and consist of a single stranded circular DNA genome, but are not well characterized (Hino, supra).

There have been attempts to use nucleic acid molecules binding to enhancer I of Hepatitis B virus DNA to modulate the replication of Hepatitis B virus (see for example US 2003/0148985 A1). However, it is so far largely unclear which cellular proteins of the host are able to regulate RNA synthesis of Hepatitis viruses. It has been suggested that morphine is able to affect the replication of Hepatitis C virus (Li Y, et al., Am J Pathol 163 (3), 2003, 1167-75), the exact mechanism of its action is unknown. Some transcription factors, LRH-1/hB1F, HNF1, HNF3b, HNF4 and C/EBP have been identified to be able to increase the activity of enhancer II region of HBV ill vitro, their exact role, if any, remains however to be identified (Cai Y N et al., Cell Res. 13 (6), 2003, 451-8).

Accordingly, it is an object of the present invention to offer an alternative method of altering the load of Hepatitis virus in a host organism based on an action different from the use of nucleoside analogues or interferon.

This object is solved by modulating the formation of such complex among others by the methods as described in the independent claims.

The present invention is thus based on the finding that a heterogeneous nuclear ribonucleoprotein (hnRNP) K or a functional fragment thereof is able to form a complex with a regulatory region on the viral genome.

This finding is in particular surprising since hnRNP K has so far been discovered to bind to a component of the nucleocapsid of Hepatitis C virus, similarly to many other cellular components (Hsieh T Y et al., J Biol. Chem. 273 (28), 1998, 17651-17659; Lai M M, Ware C F, Curr Top Microbiol Immunol 242, 2000, 117-134). The consequences of this binding have been speculated to affect the known cellular functions of hnRNP K in cells of the human body and thus to be responsible for some of the conditions caused by Hepatitis infection (Lai M M, Ware C F et al., supra).

HnRNP K is known to exist in form of different variants, which arise by alternative splicing (Dejgaard K et al., J. Mol. Biol. 236 (1), 1994, 33-48) and has a diverse set of functions. It acts as a shuttling protein, binds to various cellular factors, and acts as a transcription factor (Bomsztyk K, Denisenko O, Ostrowski J. Bioessays 26 (6), 2004, 629-638; Shawn A et al., Cell Res, 13 (6), 2003, 451-458). HnRNP K has multiple modular domains such as the K homology (KH) domains and RGG boxes that allow it to interact with both DNA and RNA. Its region especially responsible for binding to single-stranded DNA has been identified as comprising KH domain 3 (Braddock D T et al., EMBO J. 21 (13), 2002, 3476-3485; Backe P H et al., Acta Crystallogr D Biol Crystallogr 60 (Pt 4), 2004, 784-787).

The method of modulating the formation of the complex of a hnRNP K protein or a functional fragment thereof with a regulatory region on a viral genome can be used for infections caused by any Hepatitis virus. Examples of such viruses are mouse Hepatitis virus, woodchuck Hepatitis virus, ground squirrel Hepatitis virus, arctic ground squirrel Hepatitis B virus, human Hepatitis B virus (HBV), duck Hepatitis B virus, heron Hepatitis B virus, sheld goose Hepatitis B virus, snow goose Hepatitis B virus, Ross' goose Hepatitis B virus, stork Hepatitis B virus, woolly monkey Hepatitis B virus, orangutan Hepadnavirus, GB virus B, or human Hepatitis C virus (HCV). A preferred embodiment of the invention comprises the use of a hepadnavirus, in particular human Hepatitis B virus.

The corresponding Hepatitis virus may be a variant of a wild type or a known Hepatitis virus. In this connection the term “variant” refers to any form of a nucleic acid that differs in its nucleotide sequence, when compared to a corresponding known sequence. The difference can for instance be due to a polymorphism, mutations of single nucleotides, substitutions, deletions or insertions (of continuous stretches), N- and/or C-terminal additions introduced into the natural sequence.

Similarly the hnRNP K protein may be a variant of a wild type or a known hnRNP K protein. The term “variant” refers in this connection to any form of a protein that differs in its amino acid sequence, when compared to a known corresponding sequence. The difference can for instance be due to a polymorphism, changes or modifications of single nucleotides, substitutions, deletions or insertions (of continuous stretches), N- and/or C-terminal additions introduced into the natural sequence of the corresponding encoding nucleic acid sequence, alternative splicing, posttranslational modifications as well as conjugations to organic molecules of the corresponding peptide.

In this respect it should be understood that the term “a Hepatitis virus” or “a hnRNP K protein” is meant to include such variants.

The term “regulatory region” refers to any part of the Hepatitis genome that is able to stimulate or reduce the expression or amplification of the Hepatitis virus. Examples of such regions are silencers, enhancers or promoters. In one presently preferred embodiment of the invention the regulatory region is the enhancer II region of a hepadnavirus, in particular the human Hepatitis B virus.

As mentioned above the method may also comprise the use of a functional fragment of a hnRNP K protein. Such a fragment is a polypeptide that shall be defined by three criteria. Firstly, it is able to bind to and form a complex with a regulatory region of a Hepatitis virus that is stable enough to affect the replication of this Hepatitis virus. Preferably a functional fragment contains a K homology domain, in particular KH domain 3. Secondly, such a functional fragment is able to be modulated by a compound in such a way that its complex formation with a variant of a Hepatitis virus is affected. Thirdly, such a fragment may have at least 60% sequence identity with the corresponding amino acid sequence of a naturally existing variant of hnRNP K. In preferred embodiments, a respective fragment has at least 80%, most preferably at least 95% sequence identity with the corresponding amino acid sequence of a known variant of hnRNP K. The term “sequence identity” refers to the percentage of pair-wise identical residues obtained after a homology alignment of an amino acid sequence of a known hnRNP K variant with an amino acid sequence in question, wherein the percentage figure refers to the number of residues in the longer of the two sequences.

Where a method of the invention is used as an in vitro method, it may also comprise the use of a functional fragment of a Hepatitis virus. This term shall refer to a nucleic acid molecule that forms part of the respective Hepatitis virus. Three similar criteria as for a functional fragment of a hnRNP K protein shall define such nucleic acid molecule fragment. Firstly, it is able to bind to and form a complex with a hnRNP K protein that is stable enough to be detectable by at least one suitable method. In the case of a hepadnavirus a functional fragment preferably contains the enhancer II region. Secondly, such a functional fragment is able to be modulated by a compound in such a way that its complex formation with a variant of a hnRNP K protein is affected. Thirdly, such a fragment may have at least 60% sequence identity with the corresponding nucleic acid sequence of a naturally existing variant of a respective Hepatitis virus. In preferred embodiments, such a fragment has at least 75%, most preferably at least 90% sequence identity with the corresponding nucleic acid sequence of a known variant of a respective Hepatitis virus. The term “sequence identity” refers to the percentage of pair-wise identical residues obtained after a homology alignment of a nucleic acid sequence of a known variant of a respective Hepatitis virus with an nucleic acid sequence in question, wherein the percentage figure refers to the number of residues in the longer of the two sequences.

The corresponding host organism may be any species that can be potentially infected by a Hepatitis virus. Where a method of the invention is used as a screening method for the purpose of identifying or selecting compounds that are able to modulate the complex formation between a hnRNP K protein and a Hepatitis virus, the potential of the host organism of being infected may be achieved by help of additional means such as immunosuppressants or transgenic techniques. A host organism may for instance be from a mammalian or invertebrate species. Examples of mammals that may be infected are a rat, a mouse, a squirrel, a hamster, a woodchuck, an orangutan, a woolly monkey, a chimpanzee, a tamarin (saguinus oedipus), a marmoset or a human.

A method for altering the load of a Hepatitis virus in an infected host organism by modulating the formation of aforementioned complex can be performed in various ways. Generally this modulation can occur on the level of transcription or on the functional level by changing the activation state of the respective hnRNP K protein. A modulation on the level of transcription alters the amount of hnRNP K present in cells of the host organism and thus available for the complex formation with Hepatitis viruses. In this context it should be noted that typically an optimal level of the respective hnRNP K protein exists for each amount of a load of a Hepatitis virus that results in a maximal stimulation of Hepatitis virus replication. Deviating from this optimal level to both higher and lower amounts will usually result in a lower enhancement of viral replication. An example of this tendency can be found in FIG. 12. These observations may be especially of relevance when combining both modulations at the expression and the functional level. A modulation of the said complex formation on the functional level can comprise alterations of the components of the complex or a direct interference with the formation of the complex. A preferred embodiment for achieving such and other modulations with consequent effects on the said complex formation comprises administering a compound.

The compound used to modulate the said complex formation can be of any nature. It may for instance be isolated from a biological or non-biological source or chemically or biotechnologically produced. Examples for such compounds are, without being limited to, small organic molecules or bioactive polymers, such as polypeptides, for instance immunoglobulins or binding proteins with immunoglobulin-like functions, or oligonucleotides. One embodiment of such a compound is a nucleic acid molecule, in particular an RNA or DNA molecule, whereof in particular an aptamer, a Spiegelmer® (described in WO 01/92655), a micro RNA (miRNA) molecule or a small interfering RNA (si-RNA) molecule.

The use of small interfering RNAs has become a tool to “knock down” specific genes. It makes use of gene silencing or gene suppression through RNA interference (RNAi), which occurs at the posttranscriptional level and involves mRNA degradation. RNA interference represents a cellular mechanism that protects the genome. SiRNA molecules mediate the degradation of their complementary RNA by association of the siRNA with a multiple enzyme complex to form what is called the RNA-induced silencing Complex (RISC). The siRNA becomes part of RISC and is targeted to the complementary RNA species which is then cleaved. This leads to the loss of expression of the respective gene (for an overview see Sioud M, Methods Mol. Biol. 252, 2004, 1-8). A preferred embodiment of such a siRNA for the current invention comprises an in vitro synthesized molecule of 10 to 35 nucleotides, more preferably 15 to 25 nucleotides. Such siRNA molecules are long enough to cause gene suppression but not so long as to cause a sequence-nonspecific interferon response which in turn would result in a global inhibition of mRNA translation. This technology has been applied for therapeutical uses involving viruses, such as the inhibition of expression of the HIV-1 DNA (Lee N S et al., Nature Biotechnology 20 (5) 2002, 500-505). In an embodiment of the present invention siRNA molecules are used to induce a degradation of mRNA molecules encoding hnRNP K proteins. The use of siRNA is also a presently preferred embodiment for a modulation of the expression of hnRNP K.

Another example of a compound used to modulate the said complex formation is a molecule that is able to change the phosphorylation status of cellular components, in particular proteins. Examples of compounds that are known to affect the phosphorylation status of proteins are broad-spectrum kinase inhibitors, serine/threonine kinase inhibitors, tyrosine kinase inhibitors, tyrosine phosphorylation stimulators or tyrosine phosphatase inhibitors.

A preferred selection of a compound that is able to change the phosphorylation status of cellular components is a modulator of the degree of tyrosine phosphorylation of cellular proteins. This selection is based on the inventive finding that a change of the phosphorylation status of tyrosine residues in the cell has an effect on the efficiency of the complex formation of hnRNP K proteins with a regulatory region of a Hepatitis virus. This effect may be due to both a change of the phosphorylation status of tyrosine residues of hnRNP K proteins and a change of the intracellular quantity of hnRNP K proteins. The use of a compound that changes the phosphorylation status of tyrosine residues in the cell is therefore also an embodiment of a method of altering the complex formation between a hnRNP K protein and a regulatory region on the Hepatitis virus genome by means of modulating the total amount of a variant of hnRNP K in the cell.

Of the above mentioned compound groups a suitable compound identified and used in the present invention may be selected from tyrosine kinase inhibitors, a large number of which are commercially available such as tyrphostins, quinazolines, quinoxalines, quinolines, 2-phenylaminopyrimidines, flavonoids, benzo-quinoids, aminosalicylates or stilbenes (which are described in e.g. WO 9618738, WO 03035621 and references cited therein, for an example of their experimental identification see e.g. U.S. Pat. No. 6,740,665). Examples of tyrphostins are AG213, AG490, AG 879, AG 1295, AG 1478, AG 1517, AGL 2043, tyrphostin 46 and methyl 2,5-dihydroxycinnamate. Quinazolines are for instance PD153035, PD 156273, gefitinib or lapatinib; quinoxalines are for example PD153035 or ZD1839. An example for a quinoline is 5-methyl-5H-indolo[2,3-β]quinoline, an example for a 2-phenylaminopyrimidine is imatinib, examples for flavonoids are genistein or quercetin, an example for a benzoquinoid is herbimycin A, an example for an aminosalicylate is lavendustin A, and an example for a stilbene is piceatannol. Other suitable compounds may comprise a receptor tyrosine kinase inhibitor such as the tyrphostin erbstatin, an EGFR specific receptor tyrosine kinase inhibitor such as WHI-P97 or the tyrphostin AG 592, a tyrosine phosphorylation stimulator such as aurin tricarboxylic acid or a tyrosine phosphatase inhibitor such as sodium pervanadate or isoxazole carboxylic acids.

A further example of such a compound modulating the tyrosine phosphorylation of hnRNP K proteins is an agonist or antagonist for a cell surface molecule that is able to induce the regulation of a tyrosine kinase or tyrosine phosphatase. Examples of such cell surface molecules are receptor tyrosine kinases, membrane receptors with associated tyrosine kinase activity, and G protein coupled receptors, the signal transduction of which are interconnected with pathways regulating tyrosine kinases and phosphatases (see e.g. Pyne N J et al., Biochem Soc Trans. 31 (6), 2003, 1220-1225. Particularly with regard to receptor tyrosine kinases there are indications that they may affect both the binding of hnRNP K to nucleic acid molecules and the expression of hnRNP K (Ostrowski J et al., Proc. Natl. Acad. Sci. USA 98 (16), 2001, 9044-9049; Mandal M et al., J Biol. Chem. 276 (13), 2001, 9699-9704). hnRNP K is furthermore known to be phosphorylated in vivo (Dejgaard K et al., J. Mol. Biol. 236 (1), 1994, 33-48). Examples for a receptor tyrosine kinase are a receptor for a platelet derived growth factor, a receptor for erythropoietin, a receptor for tumor necrosis factor, a receptor for leukaemia inhibitory factor, a receptor for an interferon, a receptor for insulin, a receptor for an insulin-like growth factor, a receptor for an interleukin, a receptor for a fibroblast growth factor, a receptor for a granulocyte-macrophage colony stimulating factor, a receptor for a transforming growth factor, or a receptor for an epidermal growth-factor (EGF). Such receptors are known to possess the ability to phosphorylate tyrosine residues of various proteins and to be themselves able to regulate further factors inside the cell that possess a similar effect (see e.g. Pazin M J, Williams L T, Trends in Biochemical Sciences 17 (10), 1992, 374-378, for the EGF receptor see e.g. Janmaat M L, Giaccone G, Oncologist 8 (6), 2003, 576-586). The terms “agonist” and “antagonist” in this context therefore refer to the ability of the cell surface molecule to produce such effects and the modulation of this ability.

A preferred embodiment of such an agonist or antagonist is a proteinaceous molecule that binds to a molecule on the cell surface, which is able to induce the regulation of a tyrosine kinase or tyrosine phosphatase. Examples of such proteinaceous binding molecules are immunoglobulins or fragments thereof, or muteins based on a polypeptide of the lipocalin family (WO 03029462, Beste et al., Proc. Natl. Acad. Sci. USA 96, 1999, 1898-1903). Lipocalins, such as the bilin binding protein, the human neutrophil gelatinase-associated lipocalin, human Apolipoprotein D or glycodelin, posses natural ligand-binding sites that can be modified so that they bind to selected small protein regions known as haptens. Examples of other proteinaceous binding molecules are the so-called glubodies (see WO 96/23879), proteins based on the ankyrin scaffold (Hryniewicz-Jankowska A et al., Folia Histochem. Cytobiol. 40, 2002, 239-249) or crystalline scaffold (WO 01/04144, DE 199 32688) and the proteins described in Skerra, J. Mol. Recognit. 13, 2000, 167-187.

Immunoglobulins or fragments thereof are for instance known to be potentially effective receptor antagonists or agonists (see Goetzl E J et al., Immunol Lett. 93 (1), 2004, 63-69 and Debets R et al., J Immunol. 165 (9), 2000, 4950-4956 for two examples), as which they have also been used in therapy (see e.g. Cohen S A et al., Pathol Oncol Res. 6 (3), 2000, 163-174). This also applies to immunoglobulins directed against the EGF receptor (Goetzl E J et al., supra). Examples of (recombinant) immunoglobulin fragments are F_{ab} fragments, F_{V} fragments, single-chain F_{V} fragments (scFv), diabodies or domain antibodies (Holt L J et al., Trends Biotechnol. 21 (11), 2003, 484-490), all of which are well known to the person skilled in the art. It should be noted in this regard that it is well within the ability of the person skilled in the art to obtain immunoglobulins that act as an antagonist or agonist. For this purpose, classical immunization protocols according to Köhler and Milstein (Nature 256, 1975, 495-497) as well as evolutionary methods such as phage display (Brekke O H, Loset G A, Curr Opin Pharmacol. 3 (5), 2003, 544-550) with immunoglobulin fragments may be used.

For some embodiments of the invention, compounds may be used in form of a library. Examples of such libraries are collections of various small organic molecules, chemically synthesized as model compounds, or nucleic acid molecules containing a large number of sequence variants.

A compound that modulates said complex formation can be administered by any suitable means. If the host organism is a mammal, the compound may be administered parenterally or non-parenterally (enterally). In a preferred embodiment for administering to a mammal, the application ensures a delivery to blood and liver, for instance by administering a preparation of the compound orally, intravenously or by inhalation. Examples for preparations for an oral application are tablets, pills or drinking solutions, examples for preparations for intravenous administrations are injection or infusion solutions, examples of preparations for administration by inhalation are aerosol mixtures or sprays. If the host organism is a recombinant microorganism, examples of administration are the injection or addition of the compound to the environment of the microorganism. In case of the microorganism being a single cell, the latter form of administration may possibly be performed in combination with a technique that modifies the microorganism. Such a technique may comprise electroporation or a permeabilization of the cell membrane.

The method of the invention for altering the load of a Hepatitis virus present in a host organism may be used for various purposes. Examples of such purposes are therapeutic, diagnostic or test purposes. In case of a test purpose some methods may comprise the application of a compound that has already been identified as being able to modulate the complex formation of a hnRNP K protein with the enhancer II regulatory region on the HBV genome, while other methods may be directed at the identification of such compounds. In the latter embodiments of the invention the method preferably comprises measuring the number of Hepatitis virus particles in the host organism over a period of time.

The measurement of the number of Hepatitis virus particles in the host organism over a period of time can be performed by several means. Such a measurement may be performed at one or several time points after the infection with the Hepatitis virus. The detection method may comprise an amplification of the signal caused by the Hepatitis virus, such as a polymerase chain reaction (PCR) or the use of the biotin-streptavidin system, for example in form of a conjugation to an immunoglobulin. The measurement may rely on a direct or an indirect detection. An example of an indirect detection is the measurement of cellular effects, such as the measurement of cell viability or cellular replication. An example of a direct measurement is the use of an immunoglobulin, which may be conjugated to a label. In case of the host organism being a microorganism, an intracellular immunoglobulin may be used (Visintin M et al., J Immunol Methods 290 (1-2), 2004, 135-153). It should however be noted that the measurement of the amount of virus particles formed in a microorganism can be achieved by measuring the amount of virus released into the surrounding environment of such microorganism.

Methods of direct detection, also by means of commercially available kits, may include a step of complete dissociation of nucleoprotein complexes, followed by steps of nucleic acid extraction and PCR or variants of this technique such as nested PCR, or RT-PCR in the case of the nucleic acids being RNA. Subsequent steps may include electrophoresis, HPLC, flow cytometry (Mulrooney P M, Michalak T I, J Virol 77 (2), 2003, 970-979), fluorescence correlation spectroscopy (Weiner O H et al., Digestion 61 (2), 2000, 84-89) or a modified form of these techniques. A final step may be required, comprising for instance hybridization to a labelled internal probe and exposure to a film, or visualisation and quantification by staining and comparison to standard samples of known concentration, or the use of a piezoelectric nucleic acid biosensor (Zhou X et al., Journal of Pharmaceutical and Biomedical Analysis 27 (1), 2002, 341-345). Some or all of these steps may be part of an automated separation/detection system. Examples of such steps are automated real-time PCR platforms, automated viral nucleic acid isolation platforms (e.g. QIAGEN BioRobot), PCR product analyzers (e.g. Roche COBAS TaqMan) and real-time detection systems (e.g. ABI Prism 7700 or Rotor-Gene sequence detectors, Roche Amplicor monitor). Current commercially available signal amplification and detection assays include AMPLICOR HBV Monitor Test or COBAS AMPLICOR HCV Monitor Test (both Roche Molecular Diagnostics), VERSANT HBV 3.0 Assay (Bayer HealthCare—Diagnostics) or Digene Hybrid Capture II HBV DNA Test (Digene).

Where the method of the invention for altering the load of a Hepatitis virus present in a host organism is used in-vivo for the purpose of identifying compounds that are able to modulate the complex formation between hnRNP K and HBV, an advantageous embodiment of this method comprises additionally the comparison of obtained results with those of one or more control measurements.

Such a control measurement may comprise any condition that varies from the main measurement itself. Preferably it may comprise conditions of the method under which for example no viral amplification occurs or under which a complex formation between any or a certain hnRNP K protein and any or a certain Hepatitis virus cannot occur or cannot be modulated. In particular it may comprise the use of a compound that does not modulate the complex formation of a hnRNP K protein or a functional fragment thereof with the enhancer II regulatory region on the Hepatitis B virus genome.

In particularly preferred embodiments a control measurement will comprise the use of a variant of HBV that does not contain adenine (A) at position 1752 of the virus sequence. This embodiment is based on the surprising finding that a correlation exists between high levels of serum HBV DNA in an infected host and the presence of an A nucleotide at position 1752 of the virus sequence. Carriers of low levels of serum HBV DNA predominantly have a guanine (G) nucleotide at this position. Similarly, HBV fragments containing an adenine at position 1752 showed a significantly higher binding affinity for hnRNP K protein than those with a guanine, while for such fragments containing thymine or cytosine no complex formation could be detected. Examples illustrating these findings can be found in FIGS. 5 and 18.

In other embodiments of the invention the host organism is a microorganism. Such a microorganism preferably comprises a single cell. An example of a suitable microorganism expresses a recombinant Hepatitis virus and a recombinant hnRNP K, or a functional fragment thereof. A preferred embodiment of such a microorganism is a host cell that has been transformed with cloning vectors comprising nucleic acid molecules encoding a HBV and a hnRNP K variant using established standard methods. Such transformation methods may comprise one or more cell modification techniques, such as DNA injection, electroporation or magnetofection (Plank C et al., Biol. Chem. 384 (5), 2003, 737-747).

A preferred embodiment of such a microorganism is a cell derived from liver tissue, for example, but not limited to, a hepatocellular or a hepatoblastoma cell line. Examples for such cells are, but not limited to, HepG2, Hep3B, HCCM, PLC/PRF/5, Sk-Hep-1, Snu182, HuH-6 or HuH-7. With the respect to a suitable cell line it should be noted that nucleic acid molecules of Hepatitis viruses, and in particular the human Hepatitis C virus, have been found to be less cell line and species selective than a person skilled in the art would usually expect (Zhu Q et al., J Virol. 77 (17), 2003, 9204-9210; acknowledged and confirmed in Bartenschlager R, Hepatology 39 (3), 2004, 835-838). It has been observed that these viruses are able to adapt to a particular host cell environment, an effect that was speculated to be facilitated by the existence of swarms of sequence variants due to the high error rate of the viral replicase (Zhu et al., supra).

A further embodiment of a method for the identification of a compound that is able to alter the complex formation between a hnRNP K protein and a regulatory region on a Hepatitis virus genome comprises exposing the components of this complex to each other in vitro. The components, i.e. a hnRNP K protein or a functional fragment thereof and for example the nucleic acid component of a hepadnavirus or a functional fragment thereof, may be used in any suitable form. Examples are the use of one or more cell lysates or extracts containing a hnRNP K protein or a functional fragment thereof and/or HBV or a functional fragment thereof. Other examples are the use of enriched, purified or isolated hnRNP K proteins or functional fragments thereof and enriched, purified or isolated HBV, functional fragments thereof or nucleic acid molecules derived therefrom in a suitable aqueous solution. The term “enriched” means that hnRNP K proteins or functional fragments thereof constitute a significantly higher fraction of the total protein present in the cells or solution of interest than in the cells or solution from which it was taken. An enrichment may for instance comprise the isolation of a nuclear fraction from a cell extract. This may be obtained by standard techniques such as centrifugation. Examples of other means of enrichment are filtration or dialysis, which may for instance be directed at the removal of molecules below a certain molecular weight, or a precipitation using organic solvents or ammonium sulphate. A purification may for instance comprise a chromatographic technique, for example gel filtration, ion exchange chromatography, affinity purification, hydrophobic interaction chromatography or hydrophobic charge induction chromatography. Another example for a purification is an electrophoretic technique, such as preparative capillary electrophoresis. An isolation may comprise the combination of similar methods.

This embodiment of the invention, comprising the exposure of a hnRNP K protein and a Hepatitis virus or a functional fragment thereof, may, but need not rely on an amount of Hepatitis virus generated. In a preferred embodiment the method comprises a measurement of the biomolecular binding itself. Such measurements may for instance rely on spectroscopic, photochemical, photometric, fluorometric, radiological, enzymatic or thermodynamic means, or on cellular effects. An example for a spectroscopic detection method is fluorescence correlation spectroscopy (Thompson N L et al, Curr Opin Struct Biol. 12 (5), 2002, 634-641). A photochemical method is for instance photochemical cross-linking (Steen H, Jensen O N, Mass Spectrom Rev. 21 (3), 2002, 163-182). The use of photoactive, fluorescent, radioactive or enzymatic labels respectively (for an overview see: Rippe R A et al., Methods Mol. Biol. 160, 2001, 459-479) are examples for photometric, fluorometric, radiological and enzymatic detection methods. An example for a thermodynamic detection method is isothermal titration calorimetry (ITC, for an overview see: Velazquez-Campoy A et al., Methods Mol. Biol. 261, 2004, 35-54). An example of a method using cellular effects is the measurement of cell viability including its enzymatic detection or cellular replication. Some of these methods may include additional separation techniques such as electrophoresis or HPLC. In detail, examples for the use of a label comprise a compound as a probe or an immunoglobulin with an attached enzyme, the reaction catalysed by which leads to a detectable signal. An example of a method using a radioactive label and a separation by electrophoresis is an electrophoretic mobility shift assay.

Based on the finding that the presence of an adenine moiety at position 1752 of the HBV sequence is correlated to raised levels of serum HBV DNA, the present invention also refers to a method for modulating the formation of said complex for diagnostic or evaluation purposes of Hepatitis infection.

The invention is further illustrated by the following figures and non limiting examples.

### BRIEF DESCRIPTION OF THE DRAWINGS

FIG. 1 schematically illustrates the location of the enhancer II region on the Hepatitis B virus just upstream of the core promoter. This region (see also FIG. 6) has been shown to be involved in viral replication.

FIG. 2 illustrates the cloning of a full-length replication HBV construct. An available ATCC clone in pBR325 (upper) contains an EcoRI site at both ends. Primers were designed to amplify two fragments: 1-1900 and 1600-3215. A subsequent ligation using the internal EcoRI site (1/3215) ensured continuous viral Open Reading Frames. The replicative clone thus contained the promoter (1600-1900) at its 5′ end and the termination region (1600-1900) at its 3′ end. The construct was cloned into the NruI site of pcDNA3.1. Viral transcription is under its own promoter, as NruI is located outside the pCMV promoter.

FIG. 3 depicts schematically the coexpression of hnRNP K and HBV in a microorganism: a 1.4 kb RT-PCR fragment coding for the full-length hnRNP K gene of either variant 2 or variant 3 was cloned into the mammalian expression vector pcDNA 3.1 (Invitrogen). Clones of hnRNPK variants 2 and 3 had been obtained from total RNA extracted from HepG 2 cells. Their sequences corresponded to Genebank accession numbers NM_{—}031262, and NM_{—}031263 for variants 2 and 3 respectively, except that the cloned sequences contained a thymine instead of a cytosine at nucleotide position 501. The hnRNP K expression constructs were co-transfected into HepG2 cells together with a full-length replicative clone of HBV to determine the effect of hnRNPK on the replicative efficiency of the HBV construct.

FIG. 4 shows a stimulatory effect of hnRNP K on the amplification of HBV in a recombinant microorganism (FIG. 3). HepG2 cells were transfected with full-length infectious replicative HBV, hnRNPK and pcDNA3.1. Cells were harvested at 48 p.t. followed by genomic DNA extraction and HBV DNA viral load was measured. “+” and “++” indicates 3 μg and 6 μg of plasmid transfected DNA respectively.

FIG. 5 illustrates that distinct segregation between high and low viraemic HBV individuals is correlated to changes at nucleotide position 1752. Data of the corresponding DNA sequences from nucleotide 1720 to 1769 to the HBV DNA titer levels of patients are illustrated. A total of 60 patients were collated and DNA was isolated from sera and amplified with two rounds of PCR. PCR products were purified and sequenced directly to confirm the identity of the products. Results of the sequences were aligned and compared.

FIG. 6 renders the sequence of the enhancer II region of HBV. The position of the point mutation identified as being related to a change in levels of serum HBV DNA in infected donors is marked. The minimum sequence for enhancer activity has been previously defined at nucleotide 1687-1805 of the HBV genome as published at NCBI accession No NC_{—}003977 (Yee J K, Science 246, 1989, 658-661; Wang Y et al., J Virol. 64 (8), 1990, 3977-3981; Yuh C H, Ting L P, J Virol. 64 (9), 1990, 4281-4287). Variants of this enhancer have previously been linked with lower replication rates of HBV in vivo (Uchida T et al., Microbiol Immunol 38, 1994, 281-285). Some of the early publications do not refer to the sequence of the said NCBI accession No and thus deviate slightly in nucleotide counting.

FIG. 7 schematically illustrates the generation of three HBV constructs bearing guanosine, thymidine and cytidine instead of adenine at position 1752. Site-directed mutagenesis was carried out on nucleotide 1752 in the enhancer II region (1752A, 1752G, 1752T and 1752C) and amplified fragments were inserted upstream of the SV40 promoter in an enhancerless luciferase reporter vector.

FIG. 8 depicts the levels of expression activity in cells used for control measurements that express HBV variants or fragments thereof containing a base different from adenine at nucleotide position 1752. Expression levels are reflected by luciferase activity of cells expressing a vector comprising enhancer II (nucleotide positions 1686 to 1801 of NCBI accession No NC_{—}003977), a simian virus 40 (SV40) promoter, and the Luciferase gene (see example 2 below). The corresponding values are shown in the third column (“A”) and compared to values of cells expressing vectors comprising enhancer II with guanine, thymine or cytosine at position 1752 (“G”, “T”, and “C”). Constructs which do not contain adenine at position 1752 only minimally to weakly enhance the SV 40 promoter when linked to it in cis resulting in lower levels of luciferase expression as compared to an enhancer containing 1752A. The first column represents luciferase activity of an internal positive control, where a vector containing both the SV40 promoter and enhancer sequences was used, resulting in optimal luciferase expression (first column, “+”). The second column (“−”) depicts luciferase activity of the internal negative control, where a cloning of the vector itself containing of only the SV40 promoter-luciferase gene but without the enhancer element, had been done. Four different cell lines, HepG2, PLC/PRF/5 (abbreviated as “MP5” in the figure), SKHep1 and HCCM, all derived from human hepatocellular carcinomas, were used. HCCM and PLC/PRF/5 contained copies of the integrated HBV genome, while HepG2 and Sk-Hep-1 have been obtained from patients with no history of HBV infection and HBV genome integration. Cells were transiently transfected with the respective enhancer II clones (1752A, 1752G, 1752T and 1752C). In each transfection, 3 μg of DNA was transfected together with 1 μg of control/promoter luciferase DNA, harvested at 48 h p.t., and followed by luciferase activity analysis measured as relative light units (RLU) determined with a luminometer. Results of the luciferase assay were normalized to the level of the internal positive control (arbitrarily set at 100%).

FIG. 9 depicts the amino acid sequences and the corresponding encoding cDNA sequences of the known variants of hnRNP K (Genebank Accession numbers for variants 1, 2 and 3 are NM_{—}002140, NM_{—}031262, and NM_{—}031263 respectively). Variant 3 and variant 1 differ in their 5′untranslated regions (UTRs) and not in their encoding regions, hence only one example is given. Variant 2 contains a deletion of 60 bases at the end of the coding region in comparison to the other two known variants, resulting in a frame shift. Consequently its extreme C-terminus differs from the other two variants. So far no comparative studies have been published on functional differences between the two variants

FIG. 10 illustrates the naturally existing variations in form of single nucleotide polymorphisms (SNPs) in the hnRNP K gene. The hnRNP K full length cDNA from 18 normal volunteers was cloned and sequenced. The table lists all identified changes. DNA samples from subjects 5, 6, 7 and 14 to 18 did not contain any changes in comparison to the published sequence of hnRNP K (all variant 3, compare FIG. 9) and are therefore not listed. A novel SNP at nt252 was identified that involves a C-T change (subjects 9 and 10), but is non-synonymous. A deletion was observed in 2 different samples (subjects 11 and 12) in which a 15 base deletion was found just upstream of the KH3 domain (nt1108 to nt1122, nt: ATGATTATTCCTATG, SEQ ID NO: 1).

FIG. 11 summarizes the so far identified single nucleotide polymorphisms (SNPs) in the hnRNP K gene. In addition to the obtained results shown in FIG. 10, SNPs in the hnRNP K gene were extracted from Ensemble (www.ensembl.org) and Celera databases. Only two SNPs were reported from the dbSNP and both have not been validated. Furthermore a number of SNPs in the untranslated region (UTR) and intronic regions are reported in the public databases, but none have been validated (results not shown).

FIG. 12 depicts an example of a dose dependency of the two components on the complex formation between variants of hnRNP K and HBV. With a constant amount of HBV DNA used for transfection the HBV viral titer increases in a dose dependent manner up to a certain point with hnRNP K concentration, with no significant functional difference between variants 2 and 3. As a control, the empty expression vector pcDNA 3.1 does not have any effect on the HBV viral titer. HBV variants, which contain a base different from adenine at nucleotide position 1752, have 68 to 80% reduced HBV DNA when compared to the 1752A variant (left), indicating a lowered level of HBV replication. Nevertheless increasing dosage of hnRNP K is able to augment the replication efficiency of the three other variants. HepG2 cells were co-transfected with full-length replicative HBV clones, 1752A, 1752AG, 1752AT and 1752AC with increasing dosage (50, 250 and 1250 ng/ml) of hnRNP K variant 2 (v2) or variant 3 (v3) as indicated. Cells were harvested at 48 post transfection followed by genomic DNA extraction and HBV DNA viral load was measured by real-time PCR. Transfections were performed in duplicates; standard deviations are shown.

FIG. 13 A depicts all the seventeen tyrosine (Y) residues in hnRNP K. hnRNP K is being phosphorylated in vivo (Dejgaard K et al., J. Mol. Biol. 236 (1), 1994, 33-48). The binding of hnRNP K to some RNA and DNA molecules can be stimulated by the phosphorylation of tyrosine residues (Ostrowski J et al., Proc Natl Acad Sci USA, 98 (16), 2001, 9044-9049). Three tyrosine residues are of particular potential relevance to the complex formation with HBV (marked, Y72, Y449 and Y458) as they are located in the K homology (KH) domains. These regions have been identified as being involved in the interaction of hnRNP K proteins with nucleic acids.

FIG. 13 B illustrates site-directed mutagenesis on tyrosine residues (Y to F). Tyrosine residues in KH 1 and KH 3 domains are being substituted by phenylalanine (F), which cannot be phosphorylated. Arrows indicate the locations of the corresponding amino acids, which are being exchanged (Y to F) in the respective mutants M1 to M4.

FIG. 14 depicts a modulatory effect that can be achieved by a compound that alters the degree of phosphorylation hnRNP K proteins. Instead of altering the degree of phosphorylation by means of a compound, phosphorylation of specific tyrosine residues was prevented by mutating them to phenylalanine (see FIG. 13 B). This would correspond to the use of a compound that selectively inhibits phosphorylation at the corresponding sites, i.e. residues Y72, Y449 and Y458 in KH domains KH 1 and KR 3. HepG2 cells were co-transfected with HBV replicative clone 1752A and the respective mutants. “−” indicates co-transfection of HBV replicative clone 1752A with the empty expression vector pcDNA 3.1, serving as a control. The HBV viral titer increases by about 2.5 fold when cotransfected with variant 2 of hnRNPK and by about twofold when cotransfected with variant 3. Several mutants of variant 2 of hnRNPK containing a phenylalanine instead of a tyrosine at certain positions (see FIGS. 17 A and B) showed a reduced HBV viral titer. These data show that residues Y449 and Y458 in KH 3 domain may be important in the regulation of hnRNP K, a region previously identified as being responsible for binding to single-stranded DNA (Braddock D T et al., EMBO J. 21 (13), 2002, 3476-3485; Backe P H et al., Acta Crystallogr D Biol Crystallogr 60 (Pt 4), 2004, 784-787). The modulation of HBV viral load by these mutations therefore indicates that the complex formation between hnRNP K and HBV is affected by the phosphorylation of hnRNP K. These data may also reflect certain differences in complex formation between variants 2 and 3 of hnRNP K with HBV.

FIG. 15 depicts an example for an identification of an anti-EGFR immunoglobulin that modulates the complex formation between hnRNP K and HBV. A panel of different anti-EGFR immunoglobulins (Sigma, AnaSpec, Research Diagnostics) was collated and tested on two different liver cell lines, HepG2 and Huh7 that had been transfected with the HBV 1752A replicative construct (see FIG. 2). 4 μg of plasmid DNA was transfected using Lipofectamine 2000. The panel of anti-EGFR immunoglobulins were added at 6 h post-transfection as the following concentrations: Ab1 (AnaSpec, Cat # 29615): 11 μg; Ab 2 (Research Diagnostics, Cat # RDI-EGFRabS): 21.5 μg; Ab 3 (Research Diagnostics, Cat # RDI-EGFRCabrX): 2.3 mg and Ab 4 (Sigma, Cat # A204): 5 μg according to manufacturers' recommendations. Fresh aliquotes of immunoglobulins were added at 24 h post-transfection to enhance blocking effects. Cells were harvested at 48 h post-transfection, genomic DNA was extracted and measured for HBV viral load. One of the immunoglobulins (Ab2) was able to reduce HBV viral titers in both cell lines by more than threefold. This indicates that this immunoglobulin is able to inhibit signal transduction of the EGF receptor that leads to the phosphorylation hnRNP K proteins.

FIG. 16 depicts the selection of small interfering RNA (siRNA) for the modulation of the complex formation between hnRNP K and HBV, shown in FIG. 17. siRNA duplexes against hnRNP K were purchased from three different manufacturers, Dharmacon (SmartPool, “source A”), Qiagen (“source B”) and Proligo (“source C”). The first sequence of siRNA molecules of Source B (Qiagen) was GCAGUAUUCUGGAAAGUUU (SEQ ID NO: 2). This sequence was generated from the target sequence AAGCAGTATTCTGGAAAGTTT (SEQ ID NO: 3) at nucleotide positions 1366 to 1386 (designated “Target 2” in FIG. 16). The second sequence for Source B was CGAUGAAACCUAUGAUWAU (SEQ ID NO: 4), which was generated from target sequence TACGATGAAACCTATGATTAT (SEQ ID NO: 5) at nucleotide positions 688 to 708 (“Target 1” in FIG. 16). The first sequence of siRNA molecules of Source C (Proligo) was CUUGGGACUCUGCAAUAGATT (SEQ ID NO: 6), generated from target sequence AACTTGGGACTCTGCAATAGA (SEQ ID NO: 7). This target sequence corresponds to nucleotide positions 1029 to 1049 (“Target 3” in FIG. 16). The second Source C sequence, GAAUAUUAAGGCUCUCCGUTT (SEQ ID NO: 8), was generated from target sequence AAGAATATTAAGGCTCTCTCCGT (SEQ ID NO: 9) at nucleotide position 187 (“Target 1” in FIG. 20). Finally, source C sequence AGGACGUGCACAGCCUUAUTT (SEQ ID NO: 10) was generated from target sequence AAAGGACGTGCACAGCCTTAT (SEQ ID NO: 11) at nucleotide position 655 to 675 (“Target 2” in FIG. 16).

FIG. 17 depicts an example of a modulation of the complex formation between hnRNP K and HBV by means of small interfering RNA (siRNA). (I, top) HepG2 cells were co-transfected with 1752A full-length replicative HBV clone either with or without hnRNP K siRNA (2 μg, see FIG. 16). Non-targeting (“Non-T”) and lamin A/C (“Lamin”) siRNAs were used as controls. hnRNP K expression was measured 48 hours after siRNA transfection. The first two columns represent non-transfected cells and cells transfected with non-targeting siRNA. White columns represent cells co-transfected with HBV and lamin A/C siRNA. Columns on the right represent cells co-transfected with HBV and hnRNP K siRNA (A: Dharmacon, B: Qiagen, C: Proligo). (II, middle) HBV viral load was quantitated by real-time PCR in cells transfected as in (I). (III, bottom) Lamin A/C expression was measured from real-time RT-PCR. Ratios were normalized to 100% for non-transfected cells. The results represent two independent samples; standard errors of the mean are shown. HnRNP K mRNA levels as measured by quantitative real-time reverse transcription polymerase chain reaction (RT-PCR) indicate a 30% reduction relative to the non-transfected cells, non-targeting siRNA and lamin A/C siRNA controls. HBV viral load were correspondingly decreased by 50% using siRNAs from manufacturers B and C, while siRNAs from manufacturer A achieved a 15% reduction.

FIG. 18 shows an in-vitro measurement of the complex formation between fragments of HBV DNA and a hnRNP K protein in an Electrophoretic Mobility Shift Assay (EMSA). 28-mer oligonucleotide probes were designed to contain either the 1752A or 1752G nucleotide with control probes taken from the adjacent upstream sequence. EMSA was performed using HepG2 nuclear extracts with the four respective probes. Probe 1 (SEQ ID NO: 12): Lanes 1 to 4; Probe 2 (SEQ ID NO: 13, A¹⁷⁵²): Lanes 5 to 8; Probe 3 (SEQ ID NO: 14): Lanes 9 to 12; Probe 4 (SEQ ID NO: 15, G¹⁷⁵²): Lanes 13 to 16. Each set of probes contains increasing concentrations (0.0 μg, 0.05 μg, 0.10 μg and 0.15 μg) of non-specific competitor DNA of [poly-(dI)-poly-(dC)] respectively. This competitor DNA is included to minimize the binding of nonspecific proteins to the labeled probes. DNA-protein complexes migrate at a different speed than free DNA molecules. Binding of hnRNP K is therefore indicated by a signal of different mobility from the HBV DNA probe. Verification of the presence of hnRNP K as the second component in the complex can subsequently be performed by an analysis as shown in FIG. 19. hnRNP K was detected using the 1752A probe (Probe 2, lanes 5 to 8) along with a weaker band of similar size using the 1752G probe (Probe 4, lanes 13 to 16). Densitometric analysis of the bands indicated that the protein detected by Probe 2 was about 300% higher than that detected by Probe 4, suggesting that the 1752A probe has a higher binding affinity for hnRNP K.

FIG. 19 shows the identification of the complex formation between HBV and a cellular protein. 40 μg of nuclear protein extracts obtained from HepG2 cells were allowed to bind onto 5 mg DynabeadsR M-280 streptavidin-biotin-oligonucleotides in the presence of 2:1 (w/w) ratio of non-specific competitor DNA poly (dI-dC). Unbound proteins were washed out, bound proteins were eluted and loaded to 18 cm, pH 3 to 10 nonlinear Immobiline drystrips. Rehydration was carried out at constant voltage (50 V) overnight. First dimensional isoelectric focusing was followed by second dimensional vertical separation on SDS-PAGE (10%). The estimated molecular weight of the specific protein spots detected by silver staining (arrow) is indicated. It also revealed that specific protein spots appeared at a molecular weight of approximately 56 kDa.

FIG. 20 shows the identification of the cellular protein. Specific protein spots were cored out and destained according to manufacturer's instructions, following which the gel plug was alkylated with iodoacetamide and digested with trypsin. A tryptic mass map was obtained by means of Matrix-Assisted Laser Desorption/Ionization mass spectroscopy (MALDI). Sequence query of peptide fragments was carried out in Proteomic Research Services, Inc by using LC/MS/MS analysis (http://www.proteomicresearchservices.com/). Results of the 21 obtained and sequenced peptides are illustrated. Sequence alignments of the 56 kDa protein revealed high homology scores to hnRNP K proteins. Furthermore the molecular mass of the analysed protein matched that of hnRNP K proteins.

FIG. 21 depicts the amino acid sequence of the known hnRNPK variants (see also FIG. 9) and the respective regions covered by tryptic peptides (boxes) assigned by MALDI peptide mass mapping.

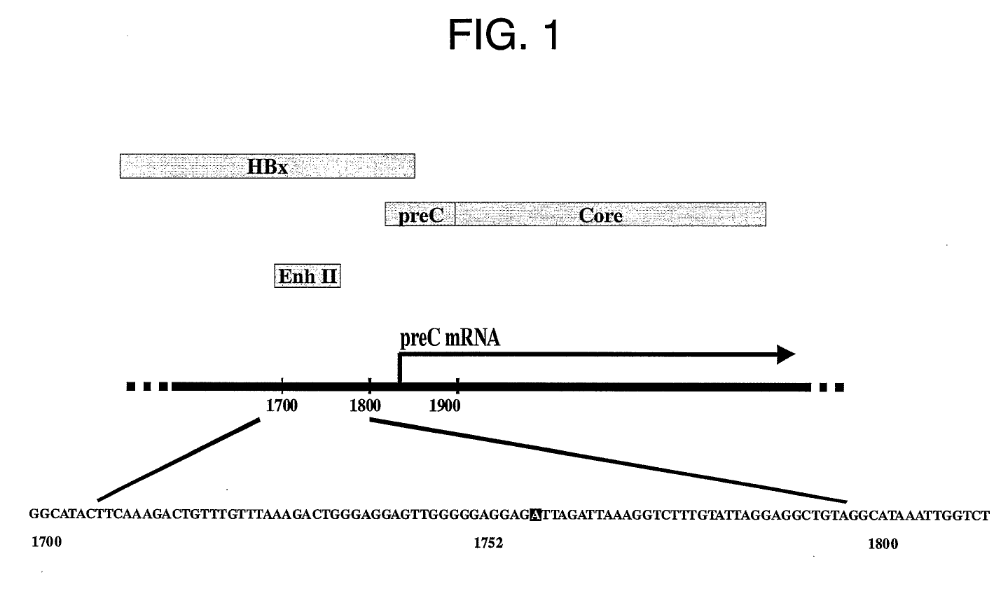

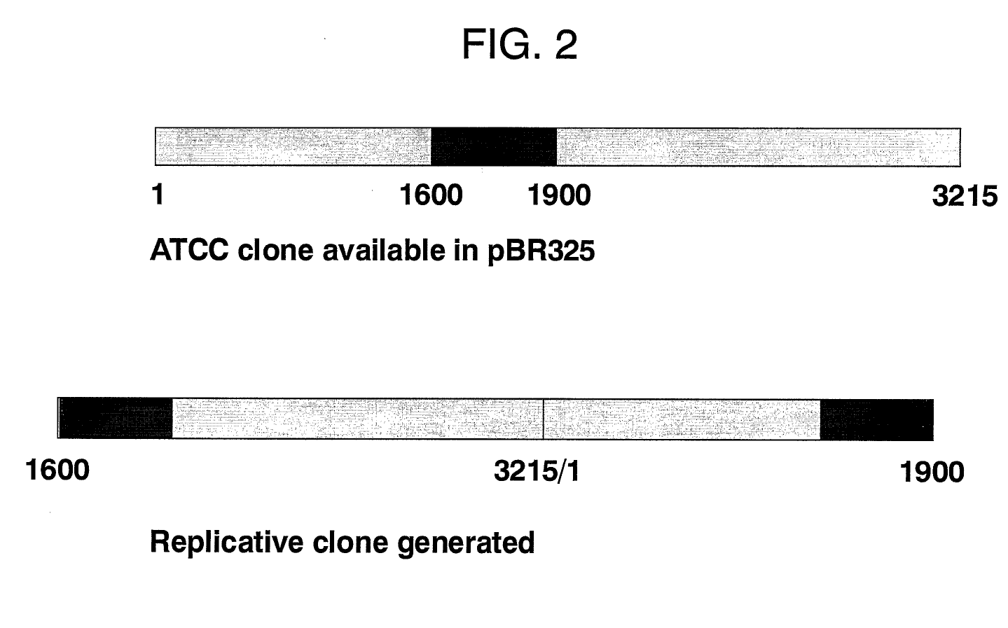

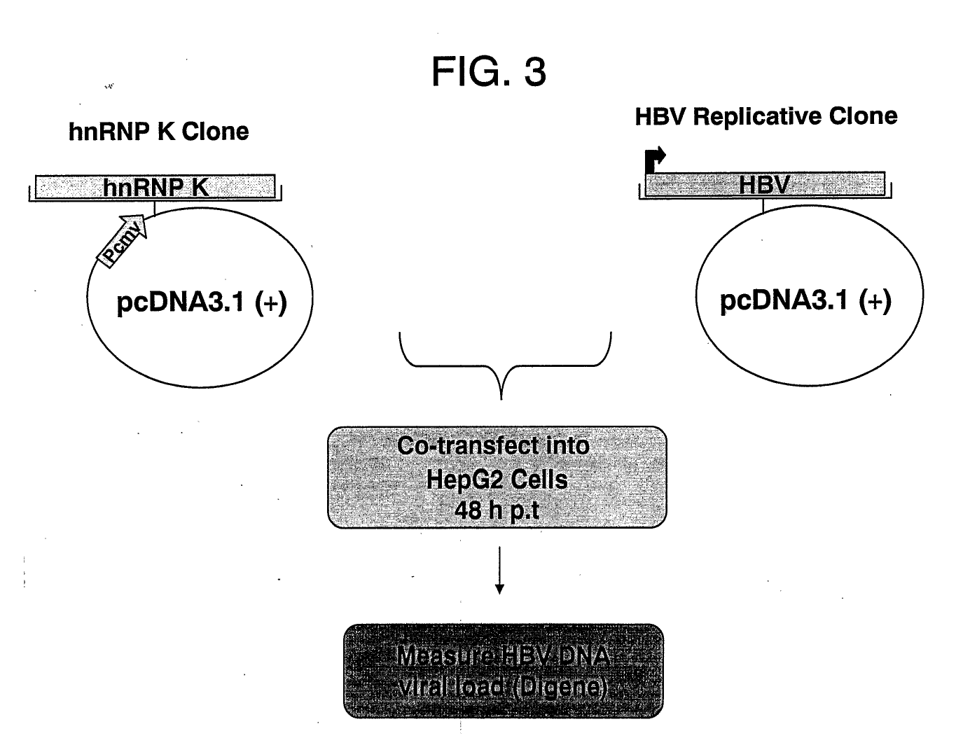

### EXAMPLES

### Example 1

### Variation of the Expression of a variant of heterogeneous nuclear Ribonucleoprotein (hnRNP) K

Unless stated otherwise, established cell culture and recombinant genetic methods were used.

HepG2 cells were cultured in complete Dulbecco's modified Eagle's medium (Invitrogen) and supplemented with 10% foetal bovine serum (Cytosystems) at 37° C. in humidified 5% CO₂.

All PCR products were generated using the Expand High Fidelity PCR System (Roche). PCR products were purified using the Qiaquick PCR purification kit (Qiagen). Ligations were done using the T4 DNA Ligase (Invitrogen). Protocols were performed according to manufacturers' instructions.

Total RNA was isolated from HepG2 with the RNeasy kit (Qiagen) according to the manufacturer's instruction. HnRNP K “variant 2” and “variant 3” clones were constructed by cloning the obtained 1.4-kb RT-PCR fragment coding for the respective hnRNP K protein. The sequences of the obtained clones corresponded to Genebank accession numbers NM_{—}031262, and NM_{—}031263 for variants 2 and 3 respectively, except that the cloned sequences contained a thymine instead of a cytosine at nucleotide position 501. The EcoR I— and Xho I-digested PCR fragments were cloned into EcoR I- and Xho I-digested pcDNA 3.1 separately. Cloning primers for “variant 2” were 5′-TAAAAGGAATTCAATATGCAAACTGAACAG-3′ (SEQ ID NO: 16) and 5′-CTAGTCCTCGAGTTAGAAAAACTTTCCAGA-3′(SEQ ID NO: 17), and cloning primers for “variant 3” were 5′-TAAAAGGAATTCAATATGCAAACTGAACAG-3′ (SEQ ID NO: 18) and 5′-CTTGCACTCGAGTTAGAATCCTTCAACATC-3′(SEQ ID NO: 19).

A full-length replicative clone of HBV 1752A was constructed using a HBV genome-containing pBR325 plasmid (ATCC, USA) as a template. Primers were designed to amplify two fragments: 1-1900 and 1600-3215. The region 1600-1900 contained the core promoter and the overlapping transcription termination region (Weiss L et al., Virology 216, 1996, 214-218). In-frame ligation of the two fragments using the internal EcoR 1 (1/3215) ensured continuous viral open reading frames were cloned into the Nru I site in pcDNA 3.1, resulting in the replicative construct. Viral transcription was under its own promoter as the Nru I site is outside the CMV IE promoter (Chen W N et al., Am. J. Gastroenterol. 95, 2000, 1098). The 1752ΔG, 1752ΔT and 1752ΔC full-length replicative clones were constructed as described for the 1752A. The first fragment, 1600-3215 was first amplified from the HBV-pBR325 plasmid and cloned into pcDNA 3.1. The 1752G, 1752T and 1752C mutations were each generated separately in the first fragment by the Quick-Change site-directed mutagenesis kit (Stratagene). Sequencing was done for verification of the constructs. The second fragment 3215 (also position 1)-1900 was then obtained from HBV-pBR325 and cloned downstream of the first fragment in pcDNA 3.1.

HepG2 cells were plated at an average density of 1×10⁶ cells per well in 35-mm tissue culture dishes and transfected with Lipofectamine 2000 (Invitrogen) according to manufacturer's instructions. Briefly, 2 mg of plasmid DNA were used for each transfection mix, and added dropwise onto the cells. After incubation for 48 h at 37° C., the cells were subsequently harvested followed by genomic DNA extraction with the DNeasy Kit (Qiagen). For control experiments, cells were transfected with the empty expression vector. Experiments were done in duplicates. After incubation for 48 h at 37° C., cells were harvested followed by genomic DNA extraction with the DNeasy Kit (Qiagen). HBV viral titer loads were measured by real-time PCR using the RealArt HBV LC PCR Kit (Artus GmbH) according to manufacturer's instructions in the LightCycler Instrument (Roche Diagnostics GmbH).

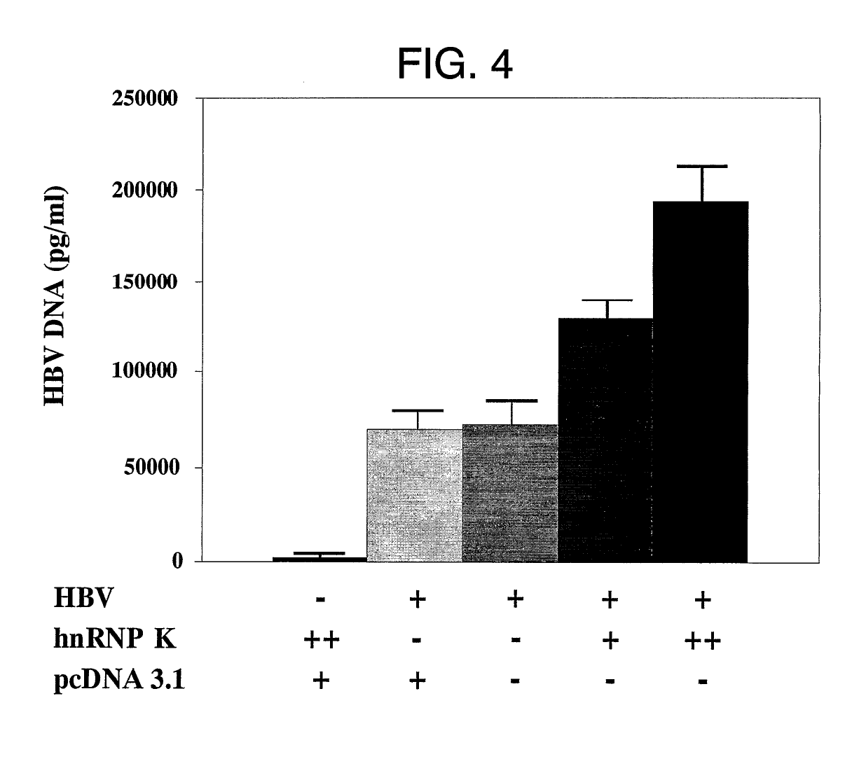

As shown in FIG. 4, the empty expression vector pcDNA 3.1 did not have any effect on the HBV viral titer when coinfected with HBV, neither did it result in any HBV detection when coinfected with hnRNPK. HBV DNA viral loads were significantly higher in cells that had been transfected with 6 μg of plasmid (++) than in cells that had been transfected with 3 μg plasmid (+).

### Example 2

### Quantification of Expression Levels of HBV Variants Containing a Base Different from Adenine at Nucleotide Position 1752

Cells of the hepatoblastoma cell lines HepG2, PLC/PRF/5, SKHep1 and HCCM were cultured in complete Dulbecco's modified Eagle's medium (Invitrogen) and supplemented with 10% foetal bovine serum (Cytosystems) at 37° C. in humidified 5% CO₂.

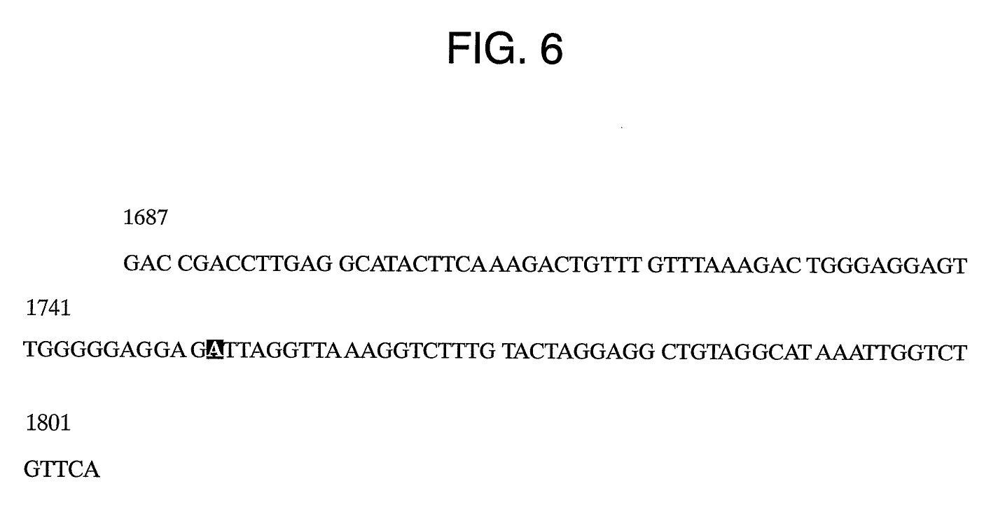

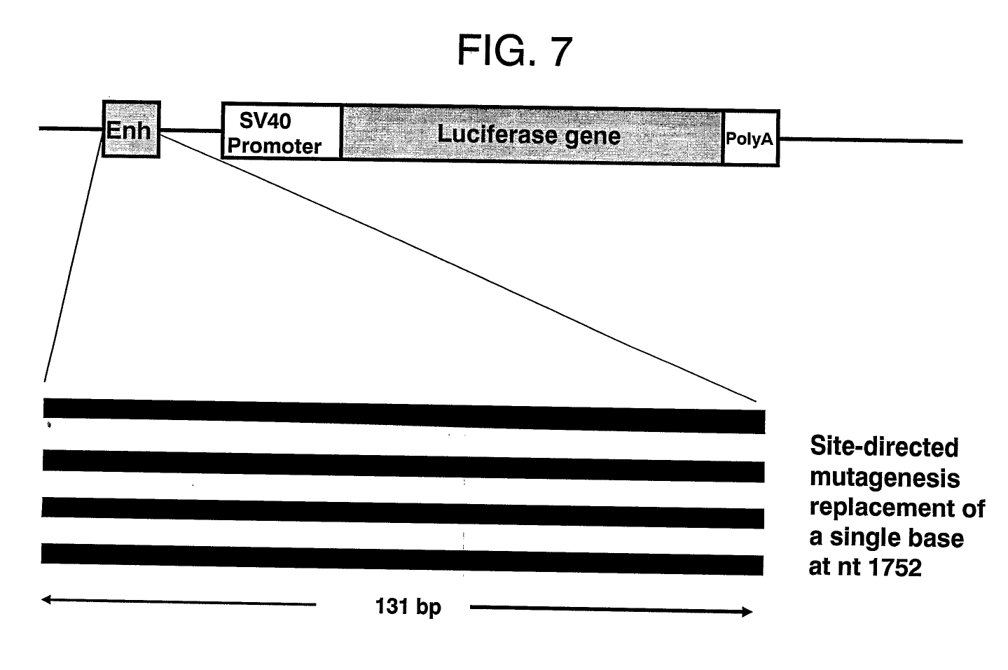

Plasmids pGL3-Control, a Luciferase plasmid with a simian virus 40 enhancer and promoter, and pGL3-Promoter, an enhancerless Luciferase plasmid with a simian virus 40 promoter upstream from the Luciferase gene, were obtained from Promega. Plasmid pGL3-Promo/A was constructed by amplifying the basic functional unit of enhancer II by PCR using primers LucF (SEQ ID NO: 20, 5′-GCACGCGTCAACGACCGACCTTGAGG-3′) and LucR (SEQ ID NO: 21, 5′-GCAGATCTACCAATTTATGCCTACAGCCTC-3′) comprising HBV nucleotide positions 1686 to 1801 (see FIG. 6). The 131 bp PCR fragment was Mlu I/Bgl II-digested and ligated with Mlu I/Bgl II-digested pGL3-Promoter. The other mutant constructs were constructed using the Gene Editor Site-Directed in vitro Mutagenesis System (Promega) to introduce the HBV Enhancer II mutations at nucleotide position 1752 (as illustrated in FIG. 7). The first mutation was mutating nucleotide A to G (pGL3Promo/G), the second was with nucleotide A to T (pGL3Promo/T), and lastly with nucleotide A to C (pGL3Promo/C). The sequences of the three mutant oligonucleotides were: 5′-GGGGGAGGAGGTTAGGTTAAA-3′ (SEQ ID NO: 22), 5′-GGGGGAGGAGTTTAGGTTAAA-3′ (SEQ ID NO: 23), and 5′-GGGGGAGGAGCTTAGGTTAAA-3′ (SEQ ID NO: 24) respectively. Constructs were sequenced for verification. HnRNP K clone was constructed by cloning a 1.4-kb RT-PCR fragment coding for the hnRNP K from total RNA extracted from HepG 2 cells. The EcoR I- and Xho I-digested PCR fragment was cloned into EcoR I- and Xho I-digested pcDNA3.1. The cloning primers were 5′-TAAAAGGAATTCAATATGCAAACTGAACAG-3′ (SEQ ID NO: 25) and 5′-CTAGTCCTCGAGTTAGAAAAACTTTCCAGA-3′ (SEQ ID NO: 26). The 1752G, 1752T and 1752C full-length replicative clones were constructed as described for the 1752A. The first fragment, 1600-3215 was first obtained from the HBV-pBR325 plasmid and cloned into pcDNA 3.1. The 1752G, 1752T and 1752C mutations were each generated separately in the first fragment by the Quick-Change site-directed mutagenesis kit (Stratagene). Sequencing was done for verification of the constructs. The second fragment 3215 (also position 1)—1900 was then amplified from HBV-pBR325 and cloned downstream of the first fragment in pcDNA 3.1.

For transfection, cells were plated at an average density of 5×10⁴ cells per well in 24-well plates and transfected with Gene Porter (Gene Therapy) according to the manufacturer's instructions. Briefly, 3 μg of plasmid DNA were diluted 1:1 with serum free medium and mixed with Gene Porter reagent, which had also been diluted 1:1 with serum free medium. After 45 min incubation at room temperature the transfection mixture was added dropwise onto the cells, which were 60-90% confluent. 3 hours post transfection fresh growth medium was added. After incubation for 48 h at 37° C., the cells were harvested and rinsed with phosphate-buffered saline (137 mM NaCl, 2.7 mM KCl, 4.3 mM Na₂HPO₄, and 1.4 mM KH₂PO₄). After genomic DNA extraction with the DNeasy Kit (Qiagen), HBV viral titer loads were measured using the Hybrid Capture II HBV DNA assay (Digene) according to the manufacturer's instructions.

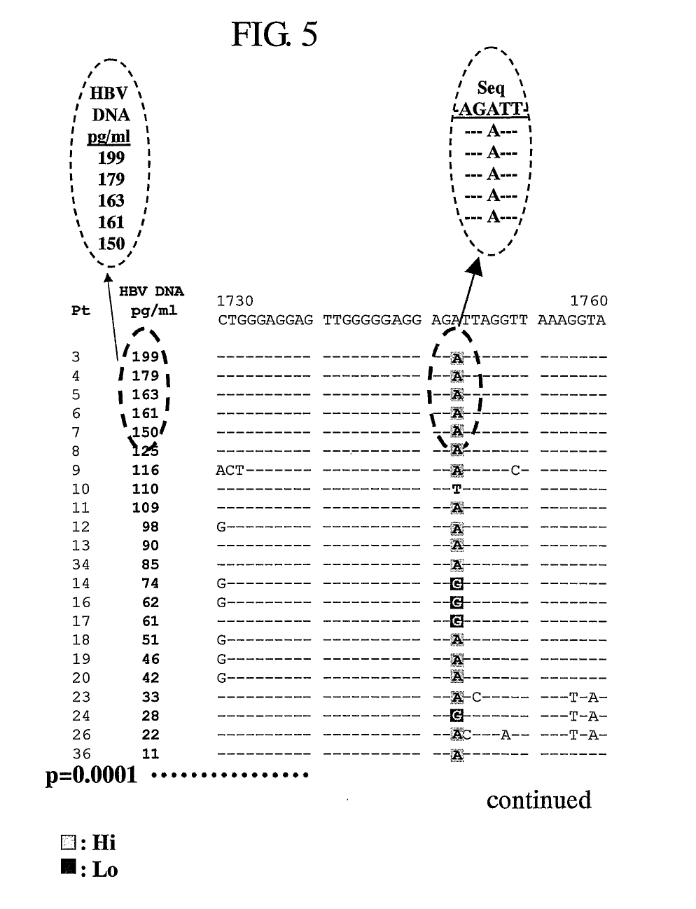

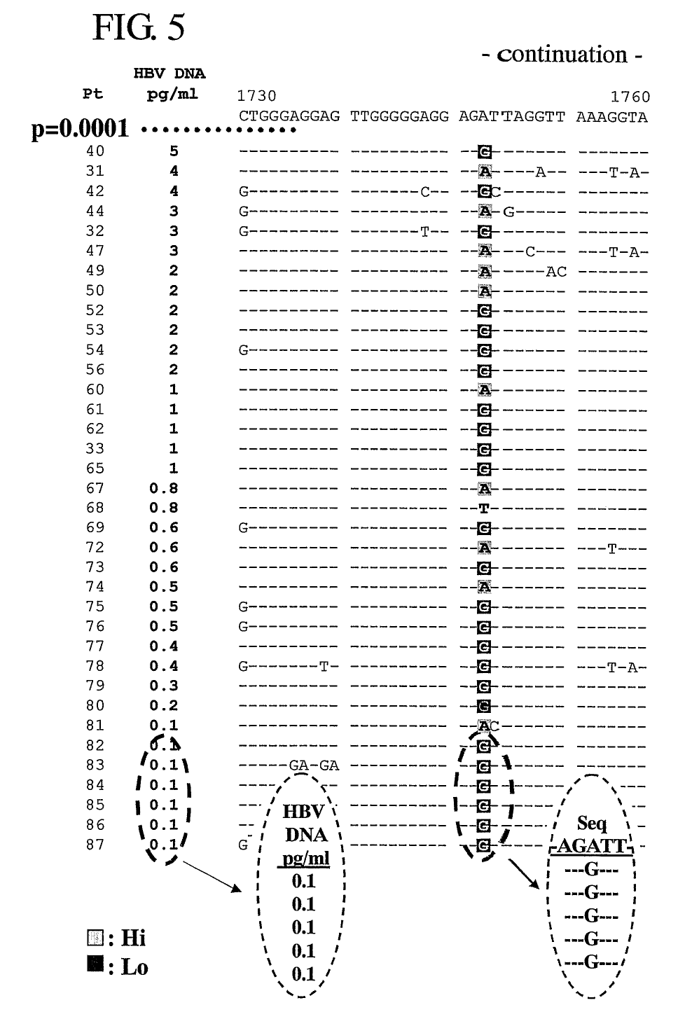

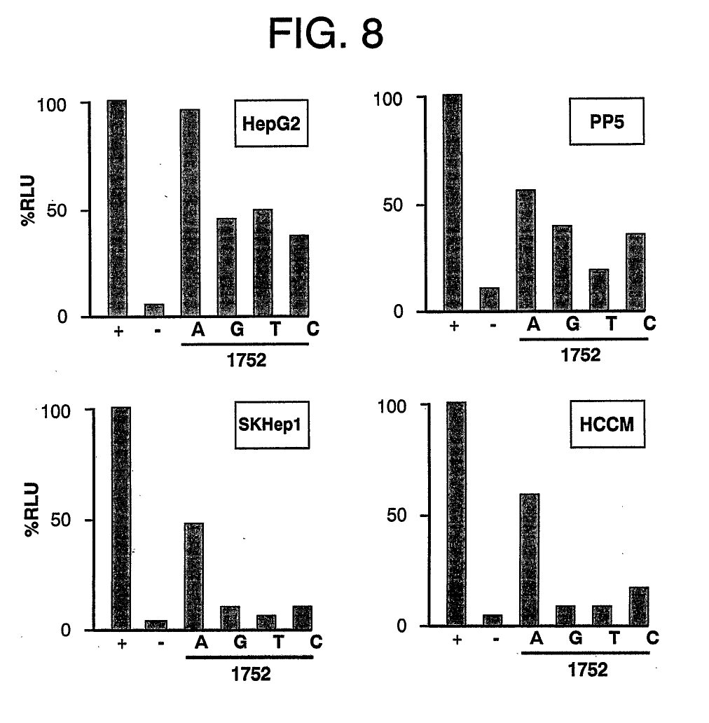

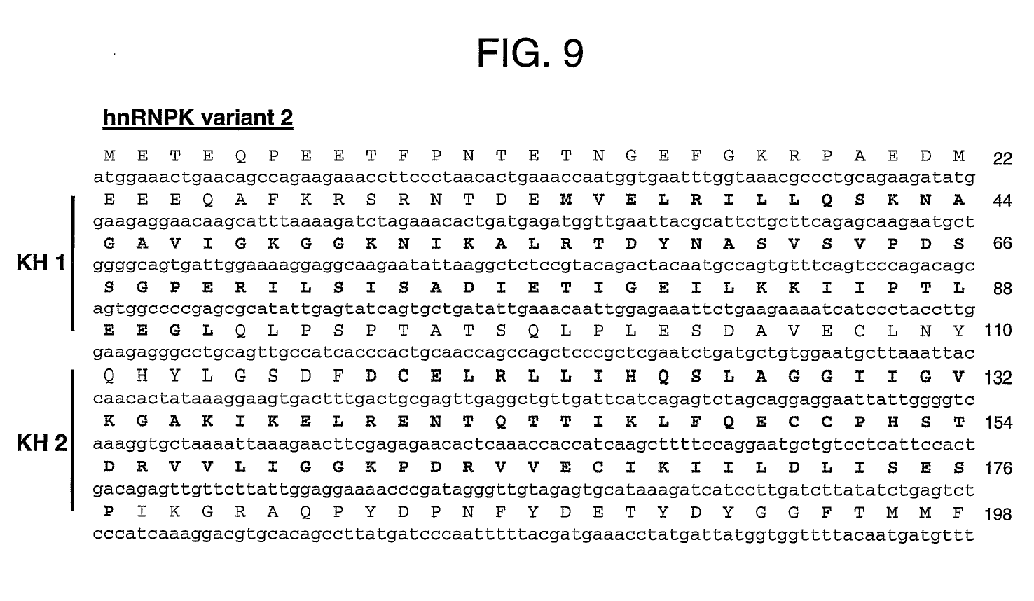

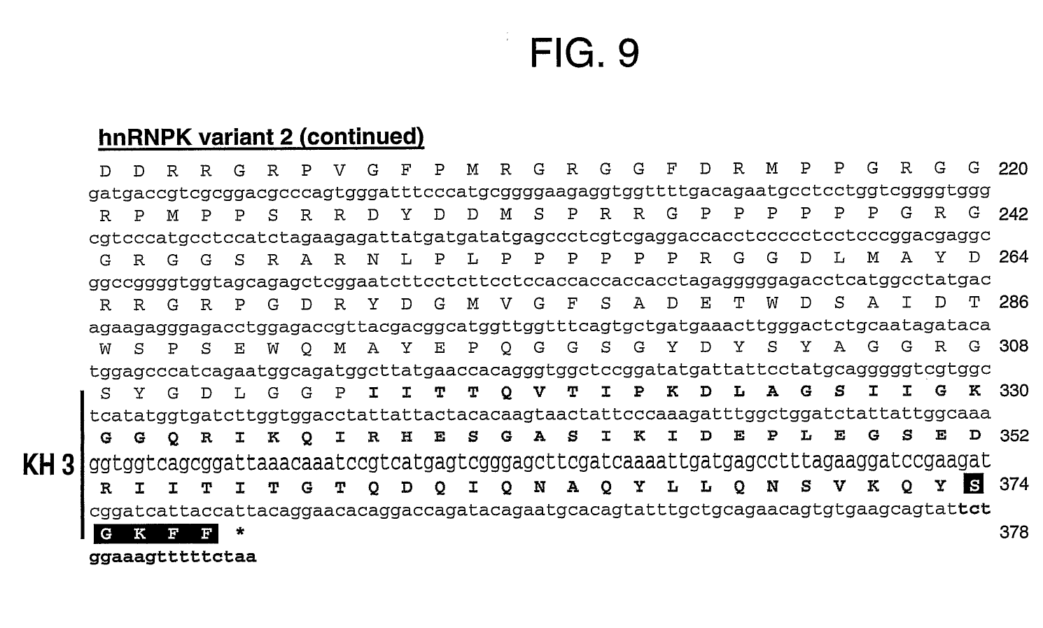

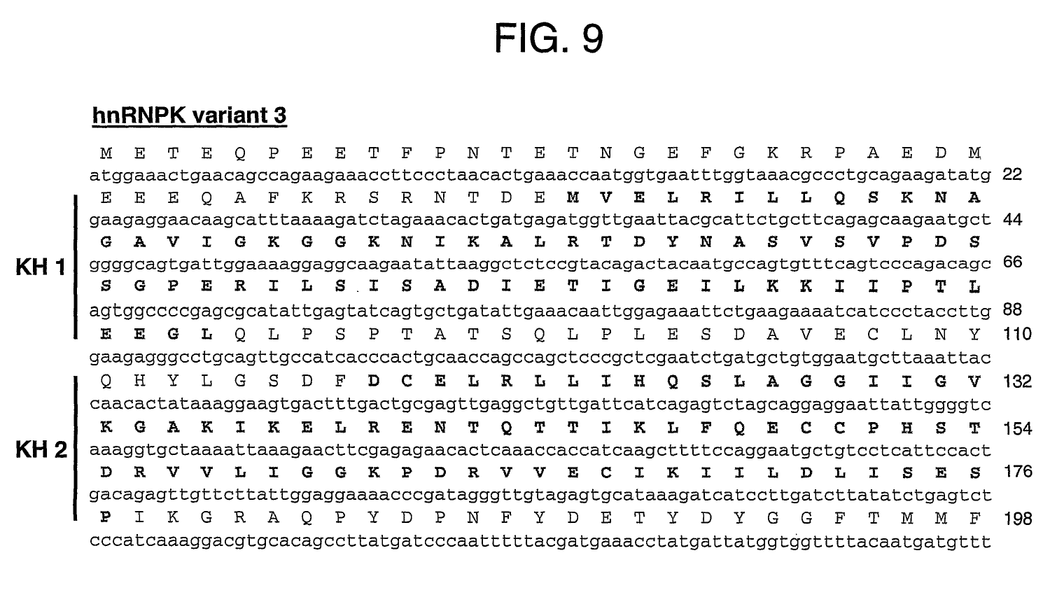

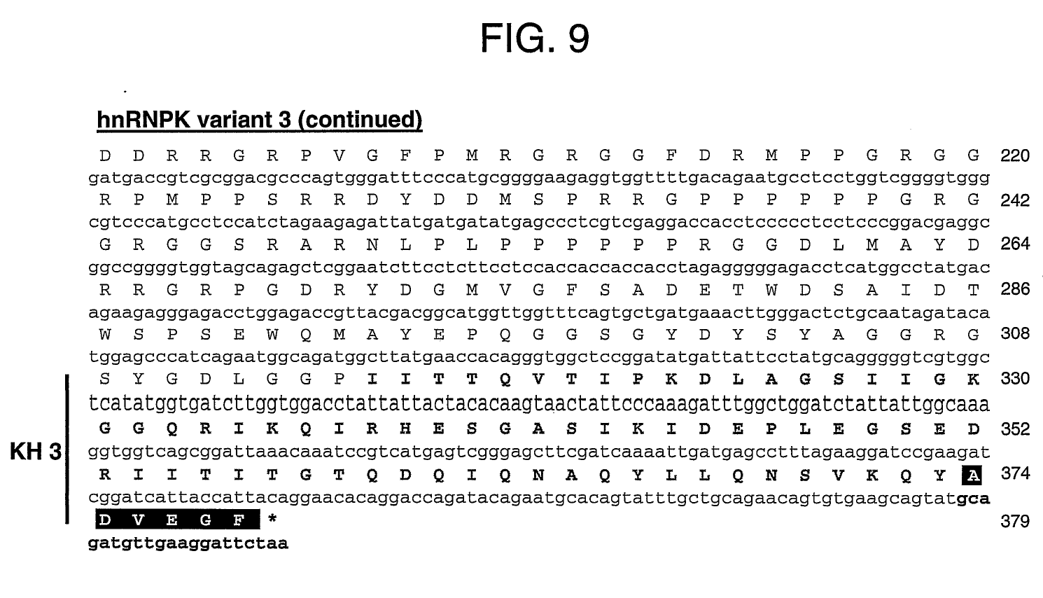

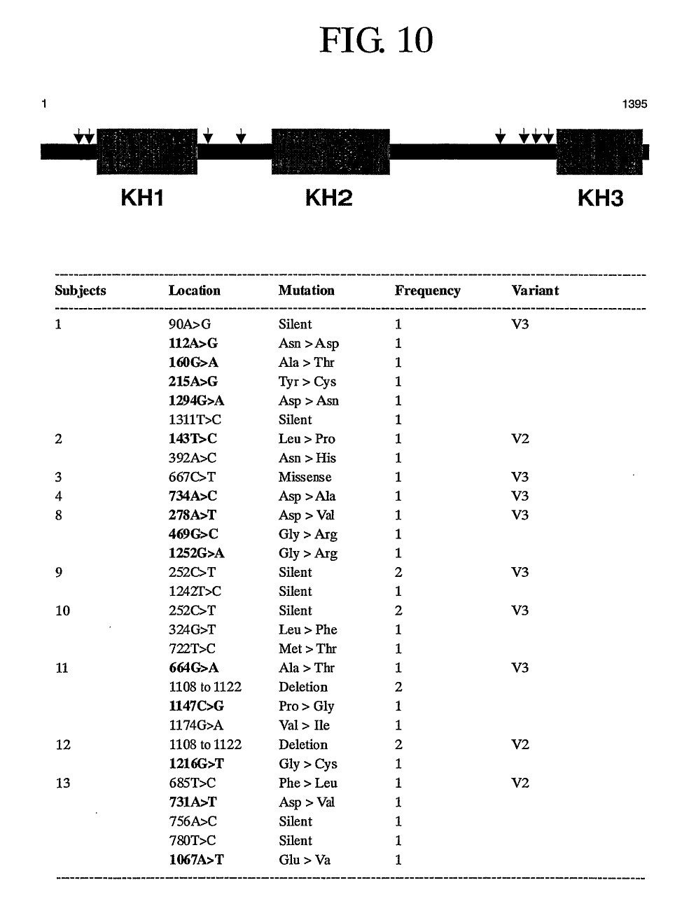

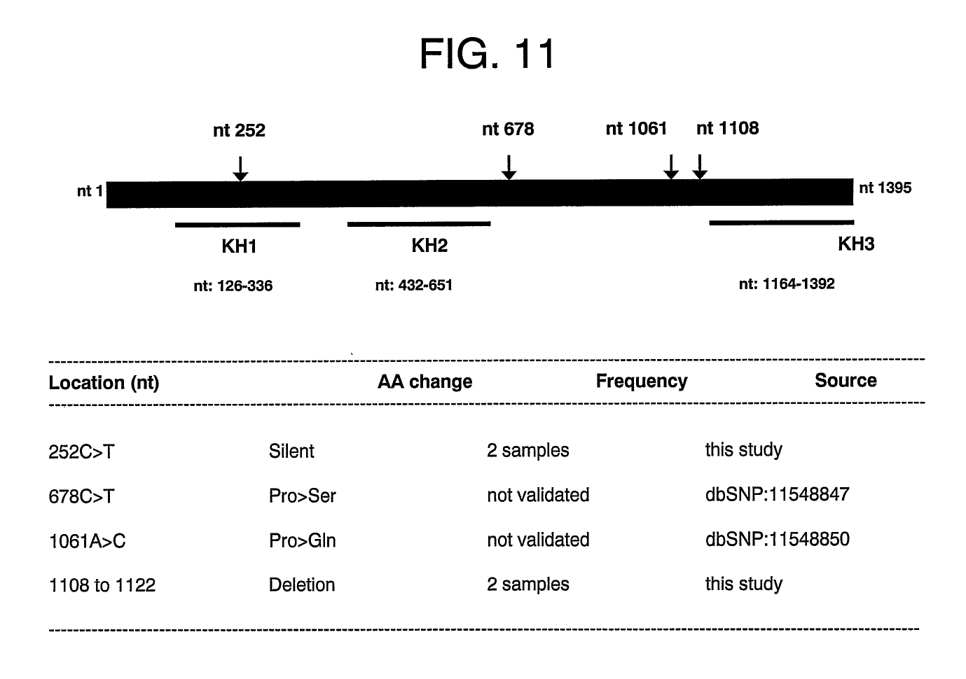

For the luciferase assays, 3 μg of plasmid DNA together with 1 μg of control/promoter luciferase plasmid-DNA were used for each transfection mix and after incubation for 48 h at 37° C., cells were harvested with Cell Culture Lysis Reagent (Promega, 25 mM Tris-phosphate ph 7.8, 2 mM DTT, 2 mM 1,2-diaminocyclohexane-N,N,N′,N′-tetraaceticacid, 10% glycerol, 1% Triton X-100). 20 μl of cell lysates were dispensed into a luminometer tube, which is subsequently placed into a Turner 20/20 luminometer (Promega). The reading was initiated by injecting 100 μl of Luciferase Assay Reagent (Promega) into the tube. Luciferase activity was measured as relative light units (RLU). Relative luciferase activity was expressed as fold increase over vector without the Enhancer element. To control for variations in transfection efficiency, experiments were performed in triplicates and repeated at least three times. For comparison results in FIG. 8 were normalized to the level of HepG2 cells that express the HBV variant with adenine at nucleotide position 1752 (set at 100% in top left, third column, “A”). HBV variants with another nucleotide at position 1752 (“G”, “T”, and “C” in FIG. 8) achieved a much lower enhancement of the SV 40 promoter as reflected in the lower levels of luciferase activity. These data also illustrate that the point mutation found in the enhancer II region by the current inventors (FIG. 5) has a significant effect on the transcriptional efficiency of enhancer II.

### Example 3

### Use of HBV Variants Containing a Base Different from Adenine at Nucleotide Position 1752 for Control Measurements

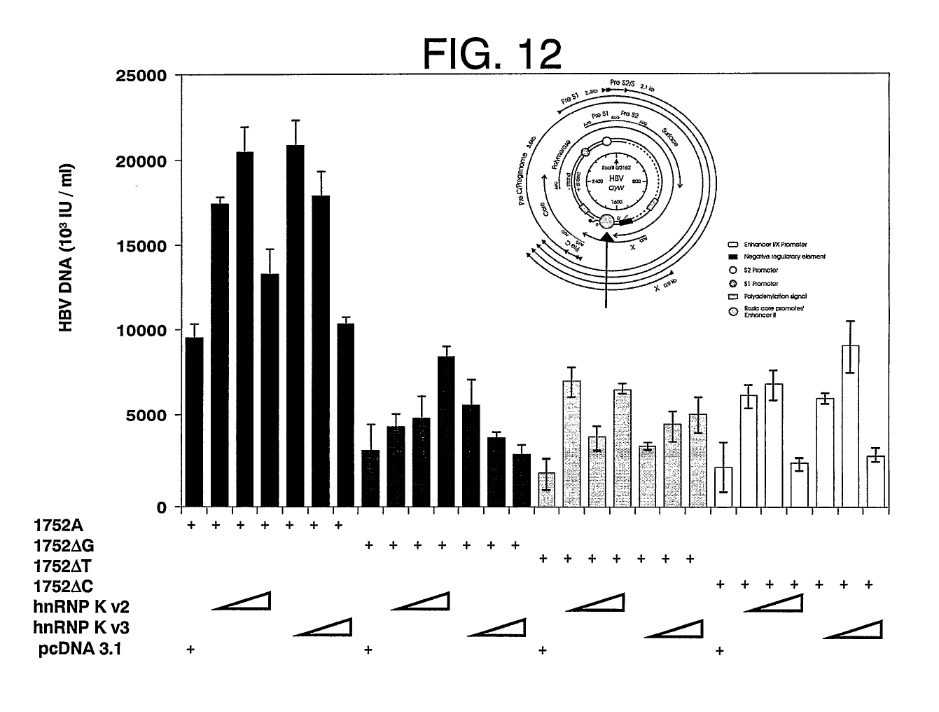

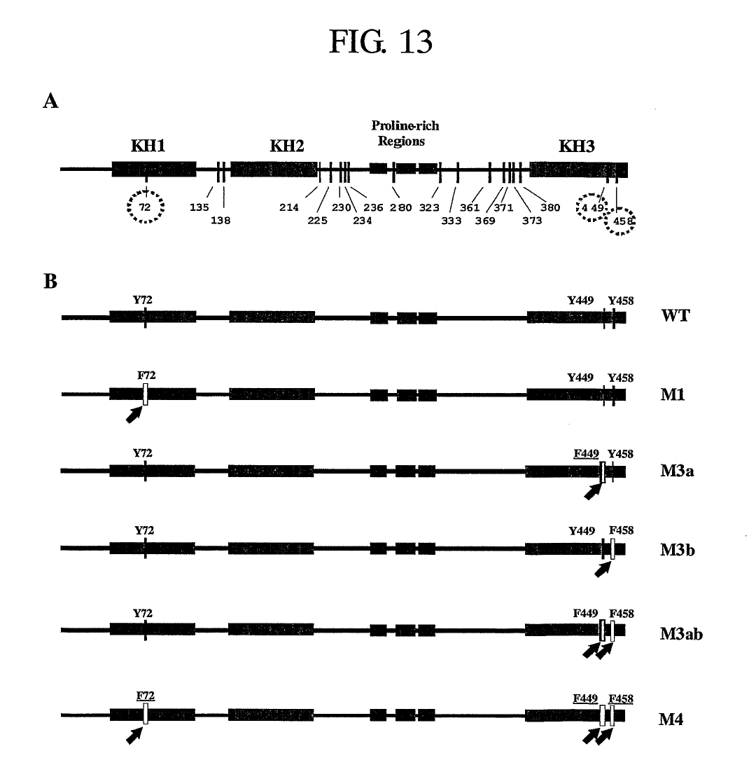

HepG2 cells were cultured in complete Dulbecco's modified Eagle's medium (Invitrogen) and supplemented with 10% foetal bovine serum (Cytosystems) at 37° C. in humidified 5% CO₂. They were plated at an average density of 1×10⁶ cells per well in 35-mm tissue culture dishes. Co-transfection with a full length 1752A replicative clone of HBV (see example 1), a respective 1752G clone, a respective 1752T clone, or a respective 1752C clone (see example 2), and either of the two hnRNP K expression constructs for “variant 2” or “variant 3” (see example 1) was performed as described in example 1. For a control experiment, the empty expression vector pcDNA 3.1 was used instead of a hnRNP K expression construct. After incubation for 48 h at 37° C., cells were harvested followed by genomic DNA extraction with the DNeasy Kit (Qiagen). HBV viral titer loads were measured by real-time PCR in the LightCycler Instrument (Roche Diagnostics GmbH) using the RealArt HBV LC PCR Kit (Artus GmbH) according to manufacturers' instructions. As predicted, the HBV viral titer increased in a dose dependent manner with hnRNP K concentration, with no significant functional difference between variants 2 and 3 (FIG. 12). The empty expression vector pcDNA 3.1, used as a control, did not have any effect on the HBV viral titer. HBV variants containing a base different from adenine at nucleotide position 1752 had 68 to 80% reduced HBV DNA when compared to the 1752A construct, indicating a lowered level of HBV replication (FIG. 12), although high dosage of hnRNP K was able to augment the replication efficiency of the three constructs. As can furthermore be seen in FIG. 12, the dose dependency of hnRNP K and HBV on their complex formation tends to reach a maximum, above which no further increase in replication efficiency can be achieved. These data also provide the definitive evidence that the inventors have mapped precisely the virus element as well as the partner host component that are needed to drive HBV replication.

### Example 4

### Modulation of the Degree of Phosphorylation of hnRNP K Proteins

Five mutants of hnRNP K variants 2 and 3, differing at Y72, Y449 and

Y458, where generated by site-directed mutagenesis. Respective tyrosine residues were exchanged to phenylalanine to mimic the effect of compounds that block phosphorylation of hnRNP K proteins. Mutants containing phenylalanine instead of tyrosine were generated using the Quick-Change site-directed mutagenesis kit (Stratagene) using the wild-type hnRNP K “variant 2” and “variant 3” clones as templates. Sequencing was done for verification of the constructs. Mutation primers used were as follows: 1. YMutF1 (SEQ ID NO: 27): Forward primer for Y72F 5′-CTC CGT ACA GAC TTT AAT GCC AGT GTT-3′; 2. YMutF2 (SEQ ID NO: 28): Reverse primer for Y72F 5′-GAC TGA AAC ACT GGC ATT AAA GTC TGT-3′; 3. YMutF3 (SEQ ID NO: 29): Forward primer for Y449F 5′-CAG AAT GCA CAG TTT TTG CTG CAG AAC-3′; 4. YMutF4 (SEQ ID NO: 30): Reverse primer for Y449F5′-CAC ACT GTT CTG CAG CAA AAA CTG TGC-3′; 5. YMutF5 (SEQ ID NO: 31): Forward primer for Y458F (for v2) 5′-AGT GTG AAG CAG TTT TCT GGA AAG TTT-3; 6. YMutF6 (SEQ ID NO: 32): Reverse primer for Y458F (for v2) 5′-TTA GAA AAA CTT TCC AGA AAA CTG CTT-3′; 7. YMutF7 (SEQ ID NO: 33): Forward primer for Y458F (for v3) 5′-AGT GTG AAG CAG TTT GCA GAT GTT GAA-3′; 8. YMutF8 (SEQ ID NO: 34): Reverse primer for Y458F (for v3) 5′-GAA TCC TTC AAC ATC TGC AAA CTG CTT-3′.

HepG2 cells were co-transfected with HBV replicative clone 1752A and the respective mutants. A control comprised co-transfection of HBV replicative clone 1752A with the empty expression vector pcDNA 3.1.

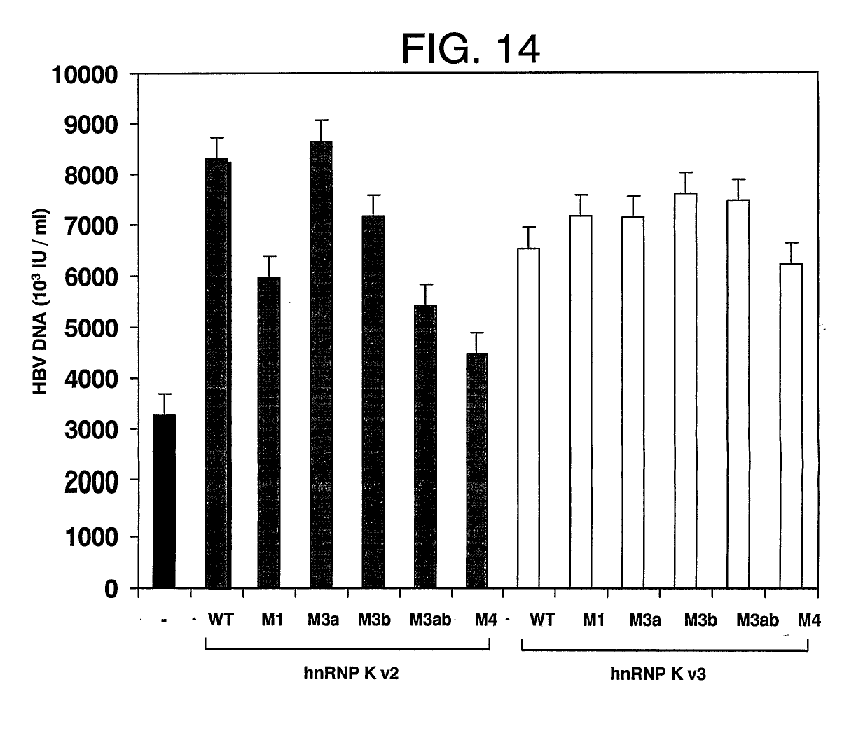

HBV viral titer loads were measured by real-time PCR using the RealArt HBV LC PCR Kit (Artus GmbH) according to manufacturer's instructions in the LightCycler Instrument (Roche Diagnostics GmbH). The kit contains reagents and enzymes for amplification of a 120-bp region of the HBV genome and for parallel detection of the specific amplification products; furthermore it contains a heterologous internal control for identification of possible PCR inhibition. HBV viral loads increased by about 2.5 fold and twofold when cotransfected with wild-type variant 2 or 3 of hnRNPK respectively (see FIG. 14). However, with several of the mutants of variant 2 of hnRNPK the increase of HBV load was lower. These data indicate that the complex formation between hnRNP K and HBV is affected by the phosphorylation of hnRNP K, especially by a phosphorylation in its KH 3 domain, where residues Y449 and Y458 are located (see FIG. 14). These results therefore also suggest that residues Y449 and Y458 in KH 3 domain may be important in the regulation of hnRNP K which then alters the upregulation of HBV viral load.

### Example 5

### Modulation of hnRNP K by an Anti-EGFR Immunoglobulin

Cells of the human hepatoma cell lines HepG2 and Huh7 were cultured in complete Dulbecco's modified Eagle's medium (Invitrogen) and supplemented with 10% foetal bovine serum (Cytosystems) at 37° C. in humidified 5% CO₂.

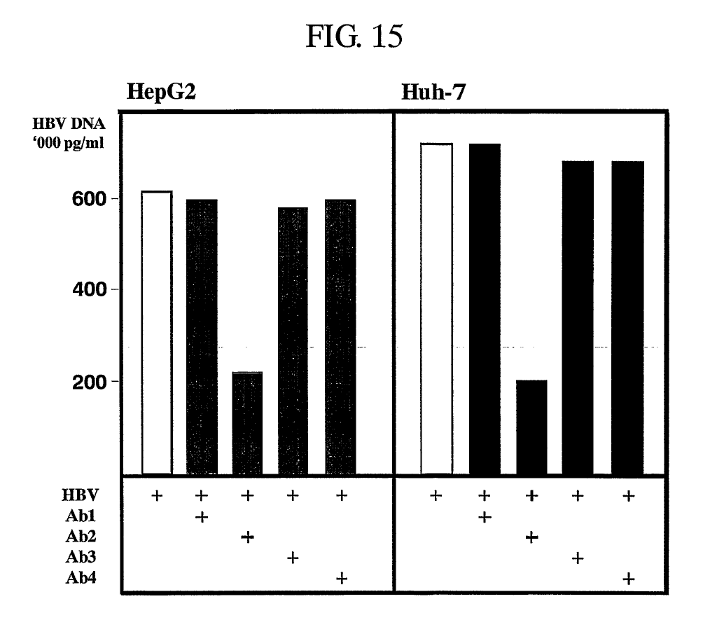

Cells were transfected with the HBV 1752A replicative construct as described in Example 1.4 μg of plasmid DNA was transfected using Lipofectamine 2000. After incubation for 6 h at 37° C. a panel of different anti-EGFR immunoglobulins (see FIG. 15) was added. Ab1 (AnaSpec, San Jose, Calif., USA Cat # 29615) is a rabbit anti-EGFR (phosphospecific) polyclonal immunoglobulin raised against a synthetic peptide corresponding to the tyrosine phosphorylated site of 1016 of human EGFR (this sequence is identical in mouse and rat origins). The immunoglobulin is supplied as an epitope affinity purified rabbit IgG, 100 μg in 200 μl phosphate buffered saline (pH7.4) containing 0.02% Proclin300. 0.22 μg/μl was used for each cell assay as shown in FIG. 15. Ab 2 (Research Diagnostics, Flanders N.J., USA, Cat # RDI-EGFRabS) is a sheep anti-EGFR immunoglobulin raised against recombinant human EGFR (partial cytoplasmic domain of EGFR inclusive of region relevant to exon 15-18). The immunoglobulin is supplied as 200 μg IgG in 200 μl Tris-HCl (pH 7.4) with 0.05% sodium azide. 0.43 μg/l was used for each cell assay. Ab 3 (Research Diagnostics, Cat # RDI-EGFRCabrX) is a rabbit immunoglobulin raised against a synthetic peptide from amino acid position 1168 to 1181 (NH2—C—S-L-D-N—β-D-Y-Q-Q-D-F—F—P—K-E-COOH), mapping to a region near the carboxy-terminus which is identical in human, mouse and rat EGFR. The amino terminal cysteine was synthesized to facilitate carrier coupling. Recognition of EGFR is independent of the phosphorylation status at tyrosine 1173. No reaction was observed against erbB-2, erbB-3 or erbB-4. The immunoglobulin is supplied as 250 μl sterile filtered neat sera with approximately 85 mg/ml protein concentration. 46 μg/μl was used for each cell assay. Ab 4 (Sigma, St. Louis, Mo., USA, Cat # A204) is a sheep immunoglobulin raised against a 20 amino acid fusion protein of the human EGFR as the immunogen. This sequence is proximal to the phosphorylation region (near the N-terminal sequence). The immunoglobulin recognizes the internal domain of the receptor molecule and will block the phosphorylation but not the binding of EGF. The immunoglobulin is supplied as a 1.3 mg/ml sterile-filtered solution in 0.15 M phosphate buffered saline (pH 7.5). 0.1 μg/μl was used for each cell assay. Fresh aliquotes of immunoglobulins were added at 24 h post-transfection to enhance blocking effects. Cells were further incubated and 48 h post-transfection harvested followed by genomic DNA extraction with the DNeasy Kit (Qiagen). HBV viral titer loads were measured by real-time PCR using the RealArt HBV LC PCR Kit (Artus GmbH) according to manufacturer's instructions in the LightCycler Instrument (Roche Diagnostics GmbH).

One anti-EGFR immunoglobulin was identified that was able to block the complex formation between hnRNP K and HBV (see FIG. 15, third bar for both cell lines). Ab 2 (Research Diagnostics, Cat # RDI-EGFRabS) reduced HBV viral titers in both cell lines by more than threefold. These data indicated that this immunoglobulin is able to inhibit signal transduction of the EGF receptor that leads to the phosphorylation hnRNP K proteins.

### Example 6

### Modulation of hnRNP K by siRNA

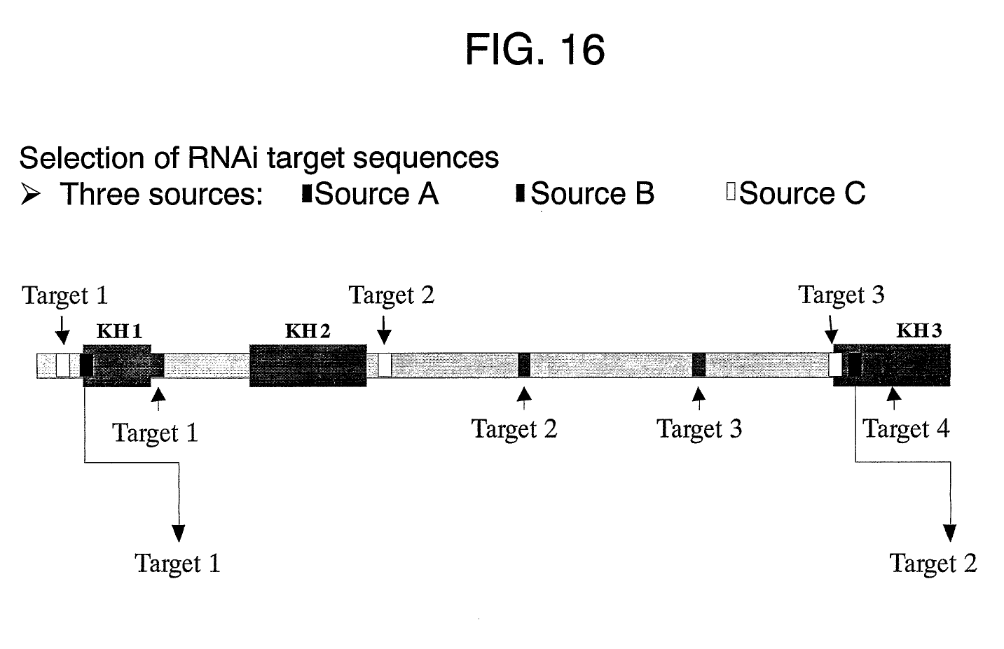

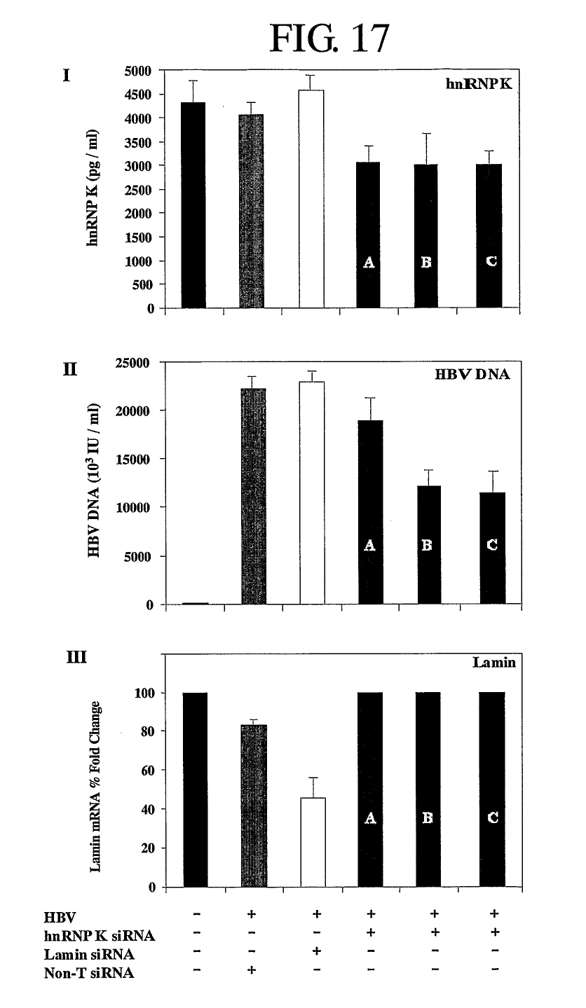

siRNA duplexes against hnRNP K were purchased from Dharmacon (SmartPool), Qiagen and Proligo. The selected target sites are illustrated in FIG. 16 and correspond to sequence AAGCAGTATTCTGGAAAGTTT (SEQ ID NO: 3, nucleotide positions 1366 to 1386, “Target 2” in FIG. 16) and TACGATGAAACCTATGATTAT (SEQ ID NO: 5, nucleotide positions 688 to 708, “Target 1” in FIG. 16) for Source B (Qiagen). The respective siRNA sequences were GCAGUAUUCUGGAAAGUUU (SEQ ID NO: 2) and CGAUGAAACCUAUGAUUAU (SEQ ID NO: 4). The first target sequence used for of Source C (Proligo) was AACTTGGGACTCTGCAATAGA (SEQ ID NO: 7), the respective siRNA sequence was CUUGGGACUCUGCAAUAGATT (SEQ ID NO: 6). This target sequence corresponds to nucleotide positions 1029 to 1049 (“Target 3” in FIG. 16). The second target sequence used for of Source C was AAGAATATTAAGGCTCTCTCCGT (SEQ ID NO: 9) at nucleotide position 187 (“Target 1” in FIG. 16). The respective siRNA sequence was GAAUAUUAAGGCUCUCCGUTT (SEQ ID NO: 8). The third Source C sequence AGGACGUGCACAGCCUUAUTT (SEQ ID NO: 10) was generated from target sequence AAAGGACGTGCACAGCCTTAT at nucleotide position 655 to 675 (“Target 2” in FIG. 16, SEQ ID NO: 11). HepG2 cells were co-transfected in 24-wells tissue culture plates with 1 mg of plasmid DNA (1752A replicative full-length clone, see example 1) and the respective siRNA duplexes (2 mg) using 6 ml Lipofectamine 2000 (Invitrogen). After 48 h, the cells were collected, and RNA and DNA extracted (Qiagen RNeasy and DNeasy kits). As controls, cells were also transfected with fluorescence labeled non-targeting siRNA to monitor the transfection efficiencies. Transfections were performed in duplicates. hnRNP K mRNA levels were measured by quantitative real-time reverse transcription polymerase chain reaction (RT-PCR). Reactions were performed using 2 ml of RNA on the LightCycler (Roche) using the RNA Master Sybr Green System (Roche) with primers 5′-AGACCGTTACGACGGCATGGT-3′ (SEQ ID NO: 35) and 5′-GATCGAAGCTCCCGACTCATG-3′ (SEQ ID NO: 36). For Lamin A/C detection, real-time reverse transcription (RT-PCR) reactions were performed using the same kit with improved primers (5′-CCCTTGCTGACTTACCGGTTC-3′, SEQ ID NO: 37, and 5′-TGCCTTCCACACCAGGTCGGT-3′, SEQ ID NO: 38) described by Lelliott et al., 2002 (Journal of Clinical Encdocrinology, 87, 728-734). Absolute quantitation of RNA was obtained by using standard curves created with in vitro-transcribed RNA by the T7 RiboMax Express in vitro transcription system (Promega). The concentration of purified transcribed RNA was measured by RiboGreen RNA quantitation reagent (Invitrogen). Serial dilutions of in vitro-transcribed RNA were prepared in duplicates. Obtained data are shown in FIG. 17. mRNA levels of hnRNP K were reduced by 30% relative to the non-transfected cells, non-targeting siRNA and lamin A/C siRNA controls. Qiagen and Proligo siRNAs (B, C in FIG. 17) reduced HBV viral loads by 50%, whereas the Dharmacori siRNAs (A) lowered HBV levels by 15%.

This difference in effectiveness of siRNA between the three sources on the replication of HBV is likely to be due to the different target regions that were selected (see FIG. 16). However, all three siRNAs apparently affect the complex formation between hnRNP K and HBV and hence HBV replication. Lamin A/C mRNA levels measured by real-time RT-PCR in HepG2 cells transfected with lamin siRNA showed a 45% reduction relative to the non-transfected cells, while non-targeting siRNA and hnRNP K siRNAs had no effect on the lamin A/C mRNA levels. Therefore lamin expression is apparently not affected by siRNA that has been selected for the modulation of the complex formation between hnRNP K and HBV. These data again also confirm that hnRNP K play a critical role in the process of HBV replication.

### Example 7

### Detection of the Interaction of HnRNP K and HBV In Vitro

In this example an enrichment of hnRNP K proteins was performed, which was achieved by preparing nuclear protein extracts as follows:

HepG2 cells (see example 1) were trypsinized, rinsed twice with ice-cold 1× phosphate-buffered saline (PBS), and incubated on ice for 10 min with 5× original packed cell volume (PCV) of buffer A [10 mM N-2-hydroxyethylpiperazine-N′-ethane sulfonic acid (HEPES) buffer (pH 7.9), 1.5 mM MgCl₂, 10 mM KCl and 1 mM dithiothreitol (DTT)]. After centrifugation at 1,000 rpm for 3 min at 4° C., cells were resuspended in 2× original PCV of buffer A and homogenized in a Dounce homogenizer with a S pestle with 10 strokes. Nuclei fractions were sedimented by a 10 min centrifugation at 2,500 rpm, resuspended in 1.5× buffer B [20 mM HEPES (pH 7.9), 0.2 mM EDTA, 1.5 mM MgCl₂, 420 mM NaCl, 0.5 mM DTT, 25% Glycerol) and treated with another 10 strokes of Dounce homogenizer. Cell suspensions were then transferred to microcentrifuge tubes and incubated for 30 min at 4° C. with gentle rotation. Nuclear debris was removed by centrifugation at 13,000 rpm for 40 min at 4° C. The supernatant was dialysed for 4 h against 2 changes of 200 ml Buffer C [20 mM HEPES pH 7.9, 0.2 mM EDTA, 20 mM MgCl₂, 20 mM KCl, 420 mM NaCl, 25% glycerol, 0.5 mM DTT, 0.5 mM phenylmethylsulfonyl fluoride (PMSF)] at 4° C. After dialysis, nuclear extracts were clarified by centrifugation at 13,000 rpm for 20 min. Nuclear extracts were then aliquoted and stored at −70° C. Protein concentration was quantitated with the Protein Assay kit (Bio-Rad Laboratories) using acetylated bovine serum albumin as a standard.

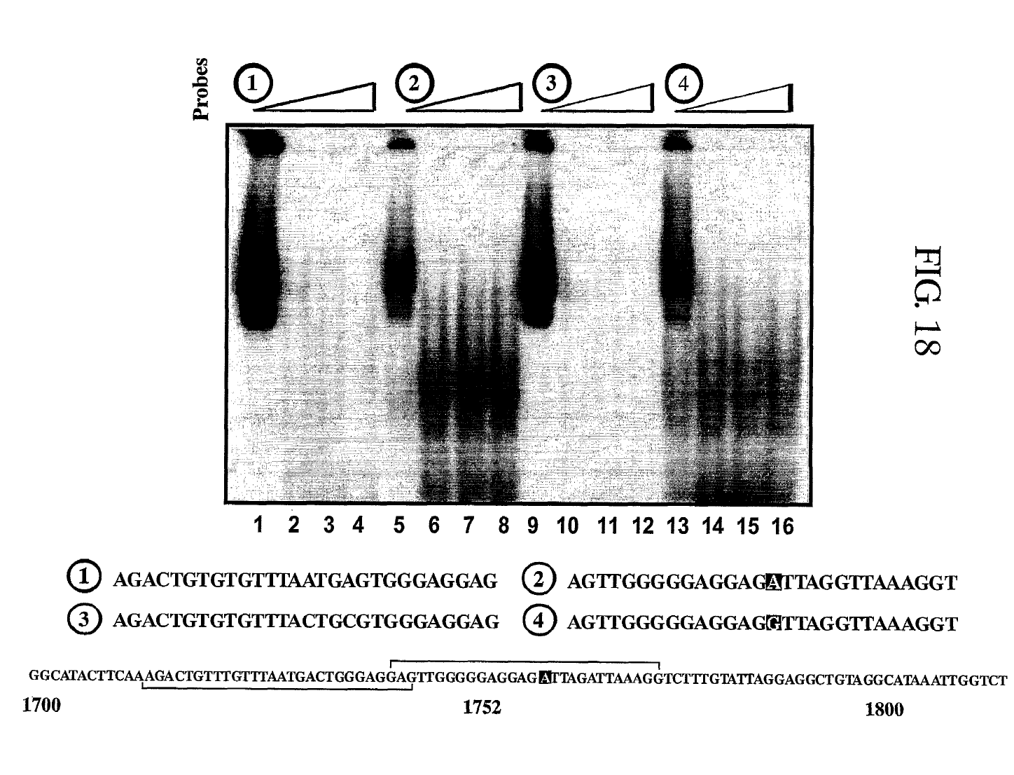

HBV-hnRNP K interaction was analysed by a method that is known to persons skilled in the art as an “electrophoretic mobility shift assay” or “EMSA”. Binding reaction procedures were performed at 37° C. for 20 min in 20 μL reaction mixtures (10 mM Tris-HCl pH 7.5, 50 mM NaCl, 1 mM EDTA and 1 mM DTT) containing 10 μg of HepG2 nuclear extracts, 0.1-0.2 μg of non-specific competitor DNA poly (dI-dC) (Amersham Pharmacia Biotech, USA) and ³²P-dATP end-labelled probe (1×10⁴ to 1×10⁵ cpm). Free DNA and DNA-protein complexes were resolved on 6% nondenaturing polyacrylamide gels. Gels were dried under vacuum at 80° C. for 1 h before exposure to X-ray film (Biomax, Kodak) at −80° C. The sequences of the oligonucleotide probes (nucleotide changes are indicated) were: Probe 1: AGACTGTGTGTTTAATGAGTGGGAGGAG (SEQ ID NO: 12); Probe 2: AGTTGGGGGAGGAGATTAGGTTAAAGGT (SEQ ID NO: 13); Probe 3: AGACTGTGTGTTTAATGCGTGGGAGGAG (SEQ ID NO: 14); Probe 4: AGTTGGGGGAGGAGGTTAGGTTAAAGGT (SEQ ID NO: 15). The obtained image is shown in FIG. 18. The 1752A probe (Probe 2, lanes 5 to 8) detected hnRNP K in form of a band. A corresponding weaker band of hnRNP was detected by the 1752G probe (Probe 4, lanes 13 to 16 in FIG. 18). A subsequent densitometric analysis of the bands indicated that 1752A probe detected an about 300% stronger signal than Probe 4. This indicates that the two probes have a correspondingly different binding affinity for hnRNP K.

### Example 8

### Identification of hnRNP K as Forming a Complex with HBV

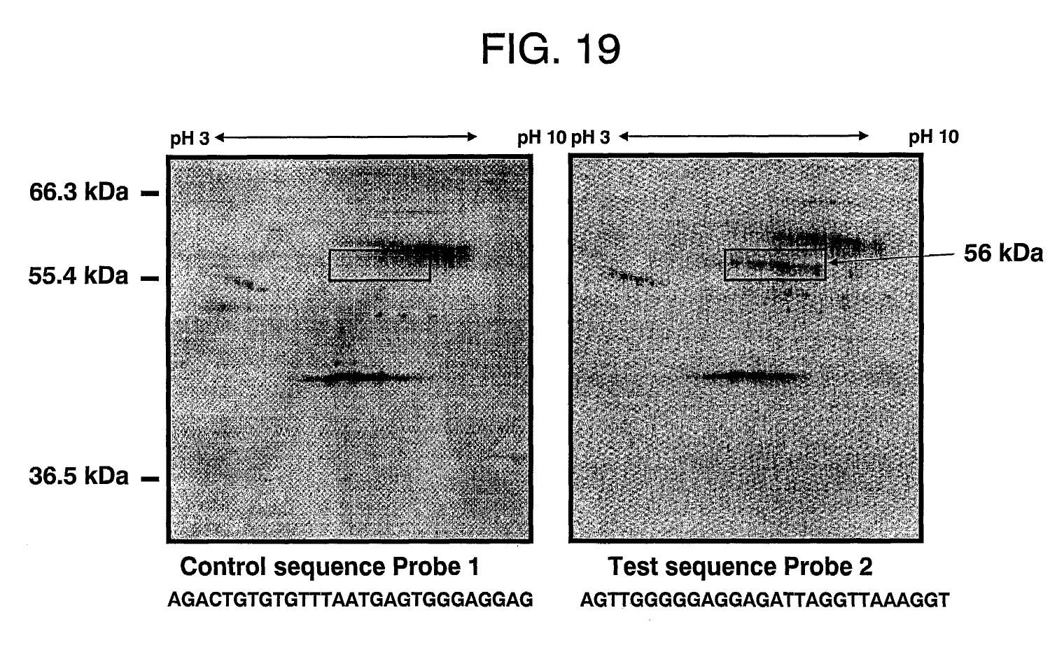

Nuclear protein extracts were obtained from the human hepatocellular carcinoma cell line HepG2 by harvesting and rinsing cells twice with ice-cold buffer A (0.15 M NaCl, 10 mM HEPES, pH 7.4), and incubation on ice for 15 min with 5× original packed cell volume of buffer B (0.33 M sucrose, 10 mM HEPES, 1 mM MgCl₂, 0.1% Triton X-100, pH 7.4). After centrifugation at 3,000 rpm for 5 min at 4° C., the pellet was washed once with buffer B and resuspended gently on ice with 200 ml of buffer C [0.45 M NaCl, 10 mM HEPES, pH 7.4, with protease inhibitor cocktail (Sigma P8340)]. The cell mixture was incubated for 15 min with gentle agitation followed by centrifugation at 13,000 rpm for 5 min. The supernatant was saved for DNA-binding proteins assay. Annealing of double-stranded oligonucleotides probes was done using 100 ml of deionised Milli Q water containing 1 nmole each of anti-sense probe and sense probe which were labeled with biotin at the 3′ end, and 5′ end, respectively. Oligonucleotide mixture solutions were heated at 95° C. for 5 min and cooled slowly to room temperature. DNA-interacting proteins were captured as described before (Gadaleta D et al., J. Biol. Chem. 271, 1996, 13537). Briefly, the oligonucleotides mixture with SEQ ID NO: 14 and SEQ ID NO: 15 (depicted in FIG. 19), was incubated with 5 mg Dynabeads® M-280 streptavidin (Dynal Biotech) at room temperature for 15 min in binding and washing buffer (5 mM Tris-HCl, 0.5 mM EDTA, 1.0 M NaCl, pH 7.5). The magnetic beads were then washed with binding and washing buffer and equilibrated with TGED buffer (20 mM Tris-HCl, 10% glycerol, 1 mM DTT, 0.01% Triton X-100, 50 mM NaCl, pH 8.0). 40 μg of extracted nuclear proteins was mixed 2:1 (w/w) with non-specific competitor DNA poly (dI-dC) (Amersham Biosciences), and adjusted to 500 μl with TGED buffer. Nuclear proteins-poly (dI-dC) solution was added to equilibrated magnetic beads-oligonucleotide probe at RT oC for 30 min. Unbound proteins were washed out with TGED buffer. Bound proteins were eluted with TGED buffer with 1 M NaCl. The same capturing and elution procedure was repeated for another four times with new aliquots of nuclear proteins-poly (dI-dC) mixture. Eluted fractions were pooled and subjected to acetone precipitation. 2-D gel electrophoresis was performed according to the Amersham Bioscience protocol with some modifications. Briefly, each sample containing acetone precipitated proteins was made up to a volume of 350 μl with rehydration buffer (7 M urea, 2 M thiourea, 4% CHAPS, 0.5% IPG buffer pH 3-10, 1.0 mg of DTT). The mixture was mixed briefly by vortexing and centrifuged at 13,000 rpm for 10 min. The supernatant was loaded to 18 cm, pH 3-10 nonlinear Immobiline DryStrips and rehydration was carried out actively at constant voltage (50 V) overnight. Isoelectric focusing (IEF) was performed using IPGphor (Amersham Biosciences) at 20° C. in stepwise mode. Briefly, strips were focused at 500 V for 1 h, 2000 V for 1 h, 5000 V for 1 h, and 8000 V for 12 h, with a total of 90 KVh accumulated. After IEF, the IPG strips were incubated for 30 min in 15 ml of SDS equilibration buffer (50 mM Tris-HCl, 6 M urea, 30% glycerol, 2% SDS, 66 mM DTT, trace amount bromophenol blue, pH 8.8), followed by second incubation with the same buffer for 30 min with iodoacetamide (375 mg/15 ml) instead of DTT. Second dimensional vertical SDS-PAGE (Protein II XL, Bio-Rad Laboratories) was carried out using 10% gels at a constant voltage of 150 V for 6-8 h at 15° C. Silver staining of the gels (SilverQuest Silver Staining Kit, Invitrogen) demonstrated positive enrichment of a specific DNA-binding protein compared with the non-specific binding control oligonucleotide probe (see FIG. 19). It also revealed that specific protein spots appeared at a molecular weight of approximately 56 kDa.

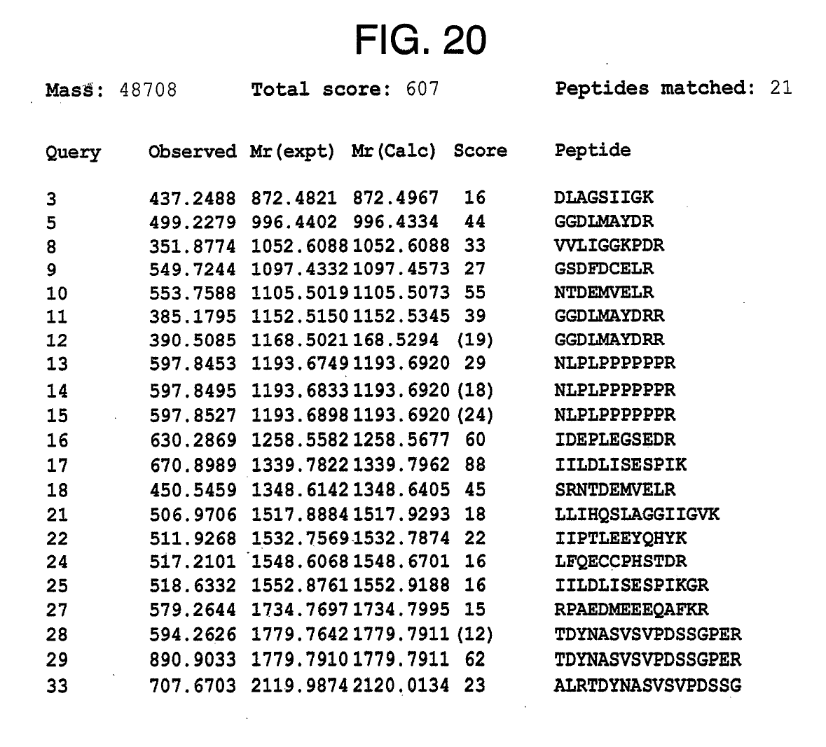

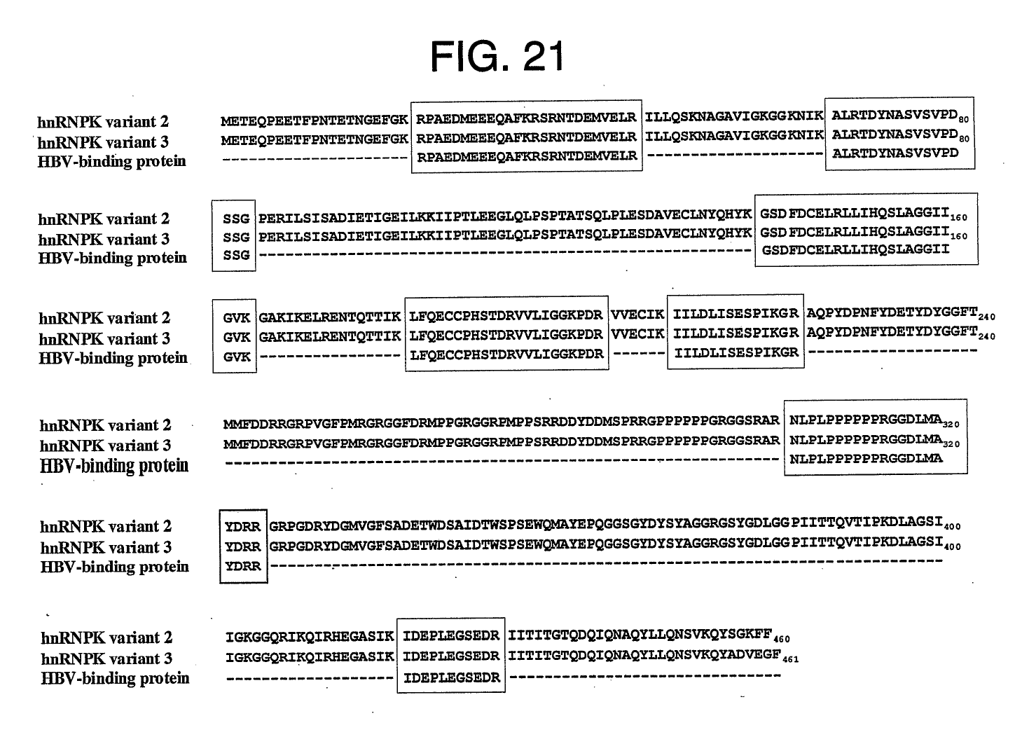

Specific protein spots were cored out and destained according to manufacturer's instructions, following which the gel plug was dried, soaked in ammonium bicarbonate solution and reduced with DTT. Alkylation was performed using iodoacetamide. Samples were digested with trypsin at 37° C. overnight on a ProGest workstation. Formic acid was added to stop the reaction. Peptides were purified with C18 Zip-tips and eluted with the matrix α-cyano-4-hydroxycinnamic acid, prepared in 60% acetonitrile, 0.2% TFA. Fifteen microliter of eluent was processed on a 75 mm C18 column at a flow-rate of 20 nl/min A tryptic mass map was obtained by means of Matrix-Assisted Laser Desorption/Ionization mass spectroscopy (MALDI.) using a Micromass Q-TOF2 mass spectrometer. Sequence query of peptide fragments was carried out in Proteomic Research Services, Inc by using LC/MS/MS analysis (http://www.proteomicresearchservices.com/). Obtained data were searched using the MASCOT search engine (www.matrixscience.com). Results of the 21 obtained and sequenced peptides are illustrated in FIG. 20. Sequence alignments of the 56 kDa protein revealed high homology scores to hnRNPK proteins. Furthermore the molecular mass of the analysed protein matched that of huRNPK proteins.

### Example 9

### Analysis and Quantification of the Complex Formation Between hnRNP K and a Regulatory Region on a Hepatitis Virus In Vitro

A method of analyzing interactions between hnRNP K and a regulatory region on a hepatitis virus, for instance the enhancer II of HBV, is known to the person skilled in the art as a “GST pull-down assay”.

The method comprises the following steps: ³⁵S-labeled enhancer II is translated in vitro according to manufacturer's instructions (TnT rabbit reticulocyte lysate system, Promega) and incubated in. 0.2 M NaCl with an excess of either Glutathione S Transferase (GST) or the fusion protein GST-hnRNP K. GST-hnRNP K may be constructed by cloning the full-length 1.4 kb hnRNP K into a GST vector using standard techniques. In a first step, GST and GST-hnRNP K vectors are transformed into DH5α, grown to an optical density at 595 nm of 0.5 and induced for 2 h with 0.2 mM Isopropyl-β-D-thiogalactopyranoside. Recombinant proteins are then purified using established chromatography techniques using Glutathione Sepharose High Performance, prepacked in GSTrap™ HP columns, according to the manufacturer's instructions (Amersham, Order No 17-5281-01). The eluate is dialyzed overnight against an aqueous buffer containing 10 mM phosphate (pH 7.5), 50 mM NaCl, 0.05% Tween, and 20% glycerol. 10 μl of in vitro translation mix are incubated with 1.8×10⁻¹⁰ mol of recombinant fusion protein in a final volume of 110 μl. After 3 h on ice, bound complexes are purified by the addition of 25 μl of a 50% (vol/vol) slurry of Glutathione Sepharose beads (Amersham, Order No 17-5279-01) in incubation buffer and mixed for 15 min at 4° C. After extensive washing with 1 ml incubation buffer, the beads are loaded onto a Laemmli gel for electrophoretical analysis. Positive results will show at least 5-fold more ³⁵S-enhancer II binding to GST-hnRNP K than to GST alone. Further verifications of this interaction can also be repeated by reciprocal experiments in which in vitro translated hnRNP K binding to GST-enhancer II.

### Example 10

### Analysis and Quantification of the Complex Formation Between hnRNP K and a Regulatory Region on a Hepatitis Virus In Vitro

A further method of analyzing interactions between hnRNP K and a regulatory region on a hepatitis virus, for instance the enhancer II of HBV, is known to the person skilled in the art as a “Chromatin immunoprecipitation (ChIP) assay” or “ChIP assay”.

HepG2 cells are transfected with the 1752A full-length replicative clone as described in example 1 and cells harvested at 48 h post-transfection. Cells are then lysed in lysis buffer (20 mM Tris-HCl pH 8, 1 mM EDTA, 1% Triton X-100, 1% SDS, 150 mM NaCl and 1 mM PMSF) and sonicated at 20% Duty for 30 sec, five to eight times. Centrifugation is done at maximum speed for 10 rain to remove cellular debris. 50 μl of sonicated sample is used and 50 μl of 10 mM Tris-Cl pH 8 added. Pronase (Roche, 20 mg/ml) is then added to samples to a final concentration of 1.5 μg/μl. After an incubation at 42° C. for 2 h and then at 65° C. overnight, LiCl is added to a final concentration of 0.8 M. Immunoprecipitation dilution buffer (20 mM Tris-Cl pH 8, 1 mM EDTA, 1% Triton X-100, 150 mM NaCl, protease inhibitors) is added to dilute the extract. 1 ml extract is mixed with 40 μl protein A sepharose beads (Protein A sepharose CL-4B, Amersham) and incubated for 15 min. The beads are then sedimented by a standard technique such as centrifugation in a table centrifuge, whereafter the supernatant is transferred to new tubes. Anti-hnRNP K immunoglobulins are added, whereafter the solution is incubated with slight agitation overnight at 4° C. Pre-equilibrated protein A beads are added, followed by an incubation for 3 h. The beads are washed and bound immunoglobulin complexes eluted with elution buffer (25 mM Tris-Cl pH 7.5, 10 mM EDTA, 0.5% SDS). DNA is extracted using the with the DNeasy Kit (Qiagen) as described in example 1. Real-time PCR as described in examples 1 and 4 is used to quantitate the amount of DNA using primers designed against the enhancer II region. This technique is illustrated in examples 1 and 4. Positive bands are detected in agarose gel using standard techniques.

1. A method for altering the load of a Hepatitis virus in a host organism infected with said virus, comprising the modulation of the complex formation of a heterogeneous nuclear ribonucleoprotein (hnRNP) K or a functional fragment thereof with a regulatory region on the Hepatitis virus genome.

2. The method of claim 1, wherein the said virus is selected from the group consisting of mouse Hepatitis virus, woodchuck Hepatitis virus, ground squirrel Hepatitis virus, arctic ground squirrel Hepatitis B virus, human Hepatitis B virus (HBV), duck Hepatitis B virus, heron Hepatitis B virus, sheld goose Hepatitis B virus, snow goose Hepatitis B virus, Ross' goose Hepatitis B virus, stork Hepatitis B virus, woolly monkey Hepatitis B virus, orangutan Hepadnavirus, GB virus B, and human Hepatitis C virus (HCV).

3. The method of claim 1, wherein the host organism is a microorganism or a mammal.

4. The method of claim 1, wherein the mammal is selected from the group consisting of a rat, a mouse, a squirrel, a hamster, a woodchuck, an orangutan, a woolly monkey, a chimpanzee, a tamarin (saguinus oedipus), a marmoset and a human.

5. The method of claim 1, wherein the modulation of said complex formation is achieved by means of altering the total amount of a variant of heterogeneous nuclear ribonucleoprotein (hnRNP) K or a functional fragment thereof in the cell.

6. The method of claim 1, comprising administering a compound that modulates the complex formation of a hnRNP K protein or a functional fragment thereof with the regulatory region on the Hepatitis virus genome.

7. The method of claim 1, wherein the regulatory region is enhancer II of a hepadnavirus.

8. The method of claim 7, wherein the enhancer II region comprises positions 1554 to 1645 of the Hepatitis B virus genome.

9. The method of claim 1, wherein the said virus is the human Hepatitis B virus.

10. The method according to claim 1, where the method is an in-vivo method for the identification of suitable compounds that modulate said complex formation.

11. The method of claim 10, comprising administering a suitable compound for modulating the complex formation of a hnRNP K or a functional fragment thereof protein with the regulatory region on the Hepatitis virus genome.

12. The method of claim 11, further comprising measuring the number of Hepatitis virus particles in the host organism over a period of time.

13. The method of claim 11 or claim 12, further comprising: comparing the obtained results with those of a control measurement.

14. The method of claim 13, wherein the control measurement comprises the use of a compound that does not modulate the complex formation of said hnRNP K protein or a functional fragment thereof with the regulatory region on the Hepatitis virus genome.

15. The method of claim 13, wherein the Hepatitis virus is the human Hepatitis B virus, the regulatory region is enhancer II, and wherein the control measurement comprises the use of a variant of HBV that does not contain adenine at position 1752 of the virus sequence.

16. The method of claim 1, wherein the host organism is a recombinant microorganism expressing a hnRNP K protein or a functional fragment thereof.

17. The method of claim 15, wherein the microorganism is a cell derived from liver tissue.

18. The method of claim 17, wherein the cell is of or derived from a hepatocellular or a hepatoblastoma cell line.

19. The method of claim 18, wherein the cell line is selected from the group consisting of HepG2, Hep3B, HCCM, PLC/PRF/5, Sk-Hep-1, Snu182, HuH-6 and HuH-7.

20. A method of claim 1, wherein the complex formation of the hnRNP K protein or a functional fragment thereof with the said regulatory region of the Hepatitis virus is reduced by means of a nucleic acid molecule.

21. The method of claim 20, wherein the nucleic acid molecule is RNA or DNA.

22. The method of claim 21, wherein the nucleic acid molecule is selected from the group consisting of an aptamer, a micro RNA (miRNA) molecule and a small interfering RNA (si-RNA) molecule.

23. The method of claim 22, wherein the nucleic acid molecule is a si-RNA molecule comprising a sequence selected from the group consisting of SEQ ID NO: 2, SEQ ID NO: 4, SEQ ID NO: 6, SEQ ID NO: 8 and SEQ ID NO: 10.

24. A method of claim 1, wherein the interaction of a hnRNP K protein or a functional fragment thereof with a regulatory region of the Hepatitis virus is modulated by a compound that modulates the phosphorylation status of cellular components.

25. The method of claim 24, wherein the compound alters the degree of phosphorylation of a hnRNP K protein or a functional fragment thereof.

26. The method of claim 24, wherein the compound alters the intracellular quantity of hnRNP K proteins or functional fragments thereof.

27. The method of claim 24, wherein the compound is an agonist or antagonist for a molecule on the cell surface.

28. The method of claim 27, wherein the molecule on the cell surface is a receptor.

29. The method of claim 28, wherein the receptor is selected from the group consisting of a receptor tyrosine kinase, a membrane receptor with associated tyrosine kinase activity, and a G protein coupled receptor.

30. The method of claim 29, wherein the receptor is selected from the group consisting of a receptor for a platelet derived growth factor, a receptor for erythropoietin, a receptor for tumor necrosis factor, a receptor for leukaemia inhibitory factor, a receptor for an interferon, a receptor for insulin, a receptor for an insulin-like growth factor, a receptor for an interleukin, a receptor for a fibroblast growth factor, a receptor for a granulocyte-macrophage colony stimulating factor, a receptor for a transforming growth factor, and a receptor for an epidermal growth-factor.

31. The method of claim 27, wherein the agonist or antagonist is a protein.

32. The method of claim 31, wherein the protein is selected from the group consisting of a mutein based on a polypeptide of the lipocalin family binding to a receptor tyrosine kinase, a glubody binding to a receptor tyrosine kinase, an immunoglobulin binding to a receptor tyrosine kinase, a protein based on the ankyrin scaffold binding to a receptor tyrosine kinase, and crystalline scaffold binding to a receptor tyrosine kinase.

33. An in-vitro method of identifying a compound capable of altering the formation of a complex between a hnRNP K protein or a functional fragment thereof, and a Hepatitis virus or a functional fragment thereof that contains the enhancer II region, comprising contacting the components that form said complex with each other.

34. The method of claim 33, comprising:
(a) adding a compound to the test tube that modulates the complex formation of said hnRNP K protein or a functional fragment thereof with the enhancer II regulatory region on the Hepatitis virus genome, and
(b) detecting the said complex formation.

35. The method of claim 34, wherein the detection is performed by a member selected from the group consisting of a suitable spectroscopic, photochemical, photometric, fluorometric, radiological, enzymatic or thermodynamic method, and a method based on cellular effects.

36. The method of claim 35, wherein the photochemical method comprises a cross-linking reaction.

37. The method of claim 35, wherein the spectroscopic method comprises the use of fluorescence correlation spectroscopy.

38. The method of claim 35, wherein the photometric detection method comprises the use of a label that is optically detectable.

39. The method of claim 35, wherein the radiological detection method comprises the use of a radioactive label.

40. The method of claim 38 that comprises the use of an electrophoretic mobility shift assay.

41. The method of claim 33 comprising the use of at least two nucleic acid molecules comprising the enhancer II region of the Hepatitis B virus DNA sequence, one of which does not contain adenine at position 1752 of the said sequence.

42. The method of claim 41, wherein the nucleic acid molecule not containing adenine at position 1752 is used for a control measurement.

43. The method of claim 33 for the in-vitro screening for potential compounds that are useful for treatment of Hepatitis infection due to their inhibition of the complex formation of a hnRNP K protein or a functional fragment thereof with a Hepatitis virus, comprising the simultaneous screening of compound libraries on multiple-well microplates using automated work stations.

44. The method of claim 43, wherein the Hepatitis infection is caused by HBV.

45. A method for treating a Hepatitis infection comprising administering to a subject a compound selected from the group consisting of aptamers, micro RNA molecules, small interfering RNA molecules, compounds that modulate the absolute quantity of hnRNK proteins in a cell, compounds that modulate the degree of phosphorylation of hnRNP K proteins, agonists for a cell surface receptor that is able to induce the regulation of a cellular kinase or phosphatase and antagonists for a cell surface receptor that is able to induce the regulation of a cellular kinase or phosphatase, wherein the viral load is altered via the modulation of the complex formation of a hnRNP K protein with a regulatory region on the Hepatitis virus genome.

46. The method of claim 45, wherein the agonist or antagonist for a cell surface receptor that is able to induce the regulation of a cellular kinase or phosphatase is selected from the group consisting of a mutein based on a polypeptide of the lipocalin family binding to a receptor tyrosine kinase, a glubody binding to a receptor tyrosine kinase, an immunoglobulin binding to a receptor tyrosine kinase, a protein based on the ankyrin scaffold binding to a receptor tyrosine kinase, a protein based on the crystalline scaffold binding to a receptor tyrosine kinase, a membrane receptor with associated tyrosine kinase activity, and a G protein coupled receptor.

47. A method for treating a Hepatitis infection comprising administering to a subject a compound identified by a method of claim 8, wherein the viral load is altered via the modulation of the complex formation of a hnRNP K protein with a regulatory region on the Hepatitis virus genome.

48. The method of claim 45, wherein the Hepatitis infection is caused by HBV.

49. A method of diagnosing a Hepatitis infection comprising using a compound selected from the group consisting of aptamers, micro RNA molecules, small interfering RNA molecules, compounds that modulate the absolute quantity of hnRNK proteins in a cell, compounds that modulate the degree of phosphorylation of hnRNP K proteins, and agonists for a cell surface receptor that is able to induce the regulation of a cellular kinase or phosphatase, antagonists for a cell surface receptor that is able to induce the regulation of a cellular kinase or phosphatase, antagonists for a cell surface receptor that is able to induce the regulation of a cellular kinase or phosphatase, wherein using said compound the viral load is altered via the modulation of the complex formation of a hnRNP K protein with a regulatory region on the Hepatitis virus genome.

50. The method of claim 49, wherein the agonist or antagonist for a cell surface receptor that is able to induce the regulation of a cellular kinase or phosphatase is selected from the group consisting of a mutein based on a polypeptide of the lipocalin family binding to a receptor tyrosine kinase, a glubody binding to a receptor tyrosine kinase, an immunoglobulin binding to a receptor tyrosine kinase, a protein based on the ankyrin scaffold binding to a receptor tyrosine kinase a protein based on the crystalline scaffold binding to a receptor tyrosine kinase, a membrane receptor with associated tyrosine kinase activity, and a G protein coupled receptor.

51. A method of diagnosing a Hepatitis infection comprising using a compound identified by a method of claim 8, wherein using said compound the viral load is altered via the modulation of the complex formation of a hnRNP K protein with a regulatory region on the Hepatitis virus genome.

52. A method of evaluating a Hepatitis infection, wherein by using at least two nucleic acid molecules comprising the enhancer II region of the Hepatitis B virus DNA sequence, one of which does not contain adenine at position 1752 of the said sequence the viral load is altered via the modulation of the complex formation of a hnRNP K protein with a regulatory region on the Hepatitis virus genome.


SEQUENCE LISTING (136 sequences)

SEQ ID NO: 1 - DNA, 15 bp, Homo sapiens
atgattattcctatg

SEQ ID NO: 2 - RNA, 19 nt, Artificial Sequence
  Description of Artificial Sequence Synthetic siRNA sequence
gcaguauucuggaaaguuu

SEQ ID NO: 3 - DNA, 21 bp, Homo sapiens
aagcagtattctggaaagttt

SEQ ID NO: 4 - RNA, 19 nt, Artificial Sequence
  Description of Artificial Sequence Synthetic siRNA sequence
cgaugaaaccuaugauuau

SEQ ID NO: 5 - DNA, 21 bp, Homo sapiens
tacgatgaaacctatgattat

SEQ ID NO: 6 - DNA, 21 bp, Artificial Sequence
  Description of Combined DNA/RNA Molecule Synthetic siRNA sequence
cuugggacucugcaauagatt

SEQ ID NO: 7 - DNA, 21 bp, Homo sapiens
aacttgggactctgcaataga

SEQ ID NO: 8 - DNA, 21 bp, Artificial Sequence
  Description of Combined DNA/RNA Molecule Synthetic siRNA sequence
gaauauuaaggcucuccgutt

SEQ ID NO: 9 - DNA, 23 bp, Homo sapiens
aagaatattaaggctctctccgt

SEQ ID NO: 10 - DNA, 21 bp, Artificial Sequence
  Description of Combined DNA/RNA Molecule Synthetic siRNA sequence
aggacgugcacagccuuautt

SEQ ID NO: 11 - DNA, 21 bp, Homo sapiens
aaaggacgtgcacagccttat

SEQ ID NO: 12 - DNA, 28 bp, Artificial Sequence
  Description of Artificial Sequence Synthetic probe
agactgtgtgtttaatgagtgggaggag

SEQ ID NO: 13 - DNA, 28 bp, Artificial Sequence
  Description of Artificial Sequence Synthetic probe
agttgggggaggagattaggttaaaggt

SEQ ID NO: 14 - DNA, 28 bp, Artificial Sequence
  Description of Artificial Sequence Synthetic probe
agactgtgtgtttaatgcgtgggaggag

SEQ ID NO: 15 - DNA, 28 bp, Artificial Sequence
  Description of Artificial Sequence Synthetic probe
agttgggggaggaggttaggttaaaggt

SEQ ID NO: 16 - DNA, 30 bp, Artificial Sequence
  Description of Artificial Sequence Synthetic primer
taaaaggaattcaatatgcaaactgaacag

SEQ ID NO: 17 - DNA, 30 bp, Artificial Sequence
  Description of Artificial Sequence Synthetic primer
ctagtcctcgagttagaaaaactttccaga

SEQ ID NO: 18 - DNA, 30 bp, Artificial Sequence; identical to SEQ ID NO: 16
  Description of Artificial Sequence Synthetic primer

SEQ ID NO: 19 - DNA, 30 bp, Artificial Sequence
  Description of Artificial Sequence Synthetic primer
cttgcactcgagttagaatccttcaacatc

SEQ ID NO: 20 - DNA, 26 bp, Artificial Sequence
  Description of Artificial Sequence Synthetic primer
gcacgcgtcaacgaccgaccttgagg

SEQ ID NO: 21 - DNA, 30 bp, Artificial Sequence
  Description of Artificial Sequence Synthetic primer
gcagatctaccaatttatgcctacagcctc

SEQ ID NO: 22 - DNA, 21 bp, Artificial Sequence
  Description of Artificial Sequence Synthetic primer
gggggaggaggttaggttaaa

SEQ ID NO: 23 - DNA, 21 bp, Artificial Sequence
  Description of Artificial Sequence Synthetic primer
gggggaggagtttaggttaaa

SEQ ID NO: 24 - DNA, 21 bp, Artificial Sequence
  Description of Artificial Sequence Synthetic primer
gggggaggagcttaggttaaa

SEQ ID NO: 25 - DNA, 30 bp, Artificial Sequence; identical to SEQ ID NO: 16
  Description of Artificial Sequence Synthetic primer

SEQ ID NO: 26 - DNA, 30 bp, Artificial Sequence; identical to SEQ ID NO: 17
  Description of Artificial Sequence Synthetic primer

SEQ ID NO: 27 - DNA, 27 bp, Artificial Sequence
  Description of Artificial Sequence Synthetic primer
ctccgtacagactttaatgccagtgtt

SEQ ID NO: 28 - DNA, 27 bp, Artificial Sequence
  Description of Artificial Sequence Synthetic primer
gactgaaacactggcattaaagtctgt

SEQ ID NO: 29 - DNA, 27 bp, Artificial Sequence
  Description of Artificial Sequence Synthetic primer
cagaatgcacagtttttgctgcagaac

SEQ ID NO: 30 - DNA, 27 bp, Artificial Sequence
  Description of Artificial Sequence Synthetic primer
cacactgttctgcagcaaaaactgtgc

SEQ ID NO: 31 - DNA, 27 bp, Artificial Sequence
  Description of Artificial Sequence Synthetic primer
agtgtgaagcagttttctggaaagttt

SEQ ID NO: 32 - DNA, 27 bp, Artificial Sequence
  Description of Artificial Sequence Synthetic primer
ttagaaaaactttccagaaaactgctt

SEQ ID NO: 33 - DNA, 27 bp, Artificial Sequence
  Description of Artificial Sequence Synthetic primer
agtgtgaagcagtttgcagatgttgaa

SEQ ID NO: 34 - DNA, 27 bp, Artificial Sequence
  Description of Artificial Sequence Synthetic primer
gaatccttcaacatctgcaaactgctt

SEQ ID NO: 35 - DNA, 21 bp, Artificial Sequence
  Description of Artificial Sequence Synthetic primer
agaccgttacgacggcatggt

SEQ ID NO: 36 - DNA, 21 bp, Artificial Sequence
  Description of Artificial Sequence Synthetic primer
gatcgaagctcccgactcatg

SEQ ID NO: 37 - DNA, 21 bp, Artificial Sequence
  Description of Artificial Sequence Synthetic primer
cccttgctgacttaccggttc

SEQ ID NO: 38 - DNA, 21 bp, Artificial Sequence
  Description of Artificial Sequence Synthetic primer
tgccttccacaccaggtcggt

SEQ ID NO: 39 - protein, 16 aa, Artificial Sequence
  Description of Artificial Sequence Synthetic peptide
CSLDNPDYQQDFFPKE

SEQ ID NO: 40 - DNA, 101 bp, Hepatitis B virus
ggcatacttcaaagactgtttgtttaaagactgggaggagttgggggaggagattagattaaaggtctttgtattaggaggctgtaggcataaattggtct

SEQ ID NO: 41 - DNA, 37 bp, Hepatitis B virus
ctgggaggagttgggggaggagattaggttaaaggta

SEQ ID NO: 42 - DNA, 37 bp, Hepatitis B virus; identical to SEQ ID NO: 41

SEQ ID NO: 43 - DNA, 37 bp, Hepatitis B virus; identical to SEQ ID NO: 41

SEQ ID NO: 44 - DNA, 37 bp, Hepatitis B virus; identical to SEQ ID NO: 41

SEQ ID NO: 45 - DNA, 37 bp, Hepatitis B virus; identical to SEQ ID NO: 41

SEQ ID NO: 46 - DNA, 37 bp, Hepatitis B virus; identical to SEQ ID NO: 41

SEQ ID NO: 47 - DNA, 37 bp, Hepatitis B virus; identical to SEQ ID NO: 41

SEQ ID NO: 48 - DNA, 37 bp, Hepatitis B virus
actggaggagttgggggaggagattaggctaaaggta

SEQ ID NO: 49 - DNA, 37 bp, Hepatitis B virus
ctgggaggagttgggggaggagtttaggttaaaggta

SEQ ID NO: 50 - DNA, 37 bp, Hepatitis B virus; identical to SEQ ID NO: 41

SEQ ID NO: 51 - DNA, 37 bp, Hepatitis B virus
gtgggaggagttgggggaggagattaggttaaaggta

SEQ ID NO: 52 - DNA, 37 bp, Hepatitis B virus; identical to SEQ ID NO: 41

SEQ ID NO: 53 - DNA, 37 bp, Hepatitis B virus; identical to SEQ ID NO: 41

SEQ ID NO: 54 - DNA, 37 bp, Hepatitis B virus
gtgggaggagttgggggaggaggttaggttaaaggta

SEQ ID NO: 55 - DNA, 37 bp, Hepatitis B virus; identical to SEQ ID NO: 54

SEQ ID NO: 56 - DNA, 37 bp, Hepatitis B virus
ctgggaggagttgggggaggaggttaggttaaaggta

SEQ ID NO: 57 - DNA, 37 bp, Hepatitis B virus; identical to SEQ ID NO: 51

SEQ ID NO: 58 - DNA, 37 bp, Hepatitis B virus; identical to SEQ ID NO: 51

SEQ ID NO: 59 - DNA, 37 bp, Hepatitis B virus; identical to SEQ ID NO: 51

SEQ ID NO: 60 - DNA, 37 bp, Hepatitis B virus
ctgggaggagttgggggaggagatcaggttaaatgaa

SEQ ID NO: 61 - DNA, 37 bp, Hepatitis B virus
ctgggaggagttgggggaggaggttaggttaaatgaa

SEQ ID NO: 62 - DNA, 37 bp, Hepatitis B virus
ctgggaggagttgggggaggagactagattaaatgaa

SEQ ID NO: 63 - DNA, 37 bp, Hepatitis B virus; identical to SEQ ID NO: 41

SEQ ID NO: 64 - DNA, 37 bp, Hepatitis B virus; identical to SEQ ID NO: 56

SEQ ID NO: 65 - DNA, 37 bp, Hepatitis B virus
ctgggaggagttgggggaggagattagattaaatgaa

SEQ ID NO: 66 - DNA, 37 bp, Hepatitis B virus
gtgggaggagttgggggcggaggctaggttaaaggta

SEQ ID NO: 67 - DNA, 37 bp, Hepatitis B virus
gtgggaggagttgggggaggagatgaggttaaaggta

SEQ ID NO: 68 - DNA, 37 bp, Hepatitis B virus
gtgggaggagttgggggtggaggttaggttaaaggta

SEQ ID NO: 69 - DNA, 37 bp, Hepatitis B virus
ctgggaggagttgggggaggagattacgttaaatgaa

SEQ ID NO: 70 - DNA, 37 bp, Hepatitis B virus
ctgggaggagttgggggaggagattaggacaaaggta

SEQ ID NO: 71 - DNA, 37 bp, Hepatitis B virus; identical to SEQ ID NO: 41

SEQ ID NO: 72 - DNA, 37 bp, Hepatitis B virus; identical to SEQ ID NO: 56

SEQ ID NO: 73 - DNA, 37 bp, Hepatitis B virus; identical to SEQ ID NO: 56

SEQ ID NO: 74 - DNA, 37 bp, Hepatitis B virus; identical to SEQ ID NO: 54

SEQ ID NO: 75 - DNA, 37 bp, Hepatitis B virus; identical to SEQ ID NO: 56

SEQ ID NO: 76 - DNA, 37 bp, Hepatitis B virus; identical to SEQ ID NO: 41

SEQ ID NO: 77 - DNA, 37 bp, Hepatitis B virus; identical to SEQ ID NO: 56

SEQ ID NO: 78 - DNA, 37 bp, Hepatitis B virus; identical to SEQ ID NO: 56

SEQ ID NO: 79 - DNA, 37 bp, Hepatitis B virus; identical to SEQ ID NO: 56

SEQ ID NO: 80 - DNA, 37 bp, Hepatitis B virus; identical to SEQ ID NO: 56

SEQ ID NO: 81 - DNA, 37 bp, Hepatitis B virus; identical to SEQ ID NO: 41

SEQ ID NO: 82 - DNA, 37 bp, Hepatitis B virus; identical to SEQ ID NO: 49

SEQ ID NO: 83 - DNA, 37 bp, Hepatitis B virus; identical to SEQ ID NO: 54

SEQ ID NO: 84 - DNA, 37 bp, Hepatitis B virus
ctgggaggagttgggggaggagattaggttaaatgta

SEQ ID NO: 85 - DNA, 37 bp, Hepatitis B virus; identical to SEQ ID NO: 56

SEQ ID NO: 86 - DNA, 37 bp, Hepatitis B virus; identical to SEQ ID NO: 41

SEQ ID NO: 87 - DNA, 37 bp, Hepatitis B virus; identical to SEQ ID NO: 54

SEQ ID NO: 88 - DNA, 37 bp, Hepatitis B virus; identical to SEQ ID NO: 54

SEQ ID NO: 89 - DNA, 37 bp, Hepatitis B virus; identical to SEQ ID NO: 56

SEQ ID NO: 90 - DNA, 37 bp, Hepatitis B virus
gtgggaggtgttgggggaggaggttaggttaaatgaa

SEQ ID NO: 91 - DNA, 37 bp, Hepatitis B virus; identical to SEQ ID NO: 56

SEQ ID NO: 92 - DNA, 37 bp, Hepatitis B virus; identical to SEQ ID NO: 56

SEQ ID NO: 93 - DNA, 37 bp, Hepatitis B virus
ctgggaggagttgggggaggagactaggttaaaggta

SEQ ID NO: 94 - DNA, 37 bp, Hepatitis B virus; identical to SEQ ID NO: 56

SEQ ID NO: 95 - DNA, 37 bp, Hepatitis B virus
ctggggaggattgggggaggaggttaggttaaaggta

SEQ ID NO: 96 - DNA, 37 bp, Hepatitis B virus; identical to SEQ ID NO: 56

SEQ ID NO: 97 - DNA, 37 bp, Hepatitis B virus; identical to SEQ ID NO: 56

SEQ ID NO: 98 - DNA, 37 bp, Hepatitis B virus; identical to SEQ ID NO: 56

SEQ ID NO: 99 - DNA, 37 bp, Hepatitis B virus; identical to SEQ ID NO: 54

SEQ ID NO: 100 - DNA, 118 bp, Hepatitis B virus
gaccgaccttgaggcatacttcaaagactgtttgtttaaagactgggaggagttgggggaggagattaggttaaaggtctttgtactaggaggctgtaggcataaattggtctgttca

SEQ ID NO: 101 - DNA, 1392 bp, Homo sapiens
  CDS (1)..(1389)
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaattaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtattctggaaagtttttctaa

SEQ ID NO: 102 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 103 - DNA, 1395 bp, Homo sapiens
  CDS (1)..(1392)
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaattaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtatgcagatgttgaaggattctaa

SEQ ID NO: 104 - protein, 464 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 105 - DNA, 28 bp, Artificial Sequence
  Description of Artificial Sequence Synthetic probe
agactgtgtgtttactgcgtgggaggag

SEQ ID NO: 106 - DNA, 101 bp, Hepatitis B virus
ggcatacttcaaagactgtttgtttaatgactgggaggagttgggggaggagattagattaaaggtctttgtattaggaggctgtaggcataaattggtct

SEQ ID NO: 107 - protein, 9 aa, Artificial Sequence
  Description of Artificial Sequence Synthetic peptide
DLAGSIIGK

SEQ ID NO: 108 - protein, 9 aa, Artificial Sequence
  Description of Artificial Sequence Synthetic peptide
GGDLMAYDR

SEQ ID NO: 109 - protein, 10 aa, Artificial Sequence
  Description of Artificial Sequence Synthetic peptide
VVLIGGKPDR

SEQ ID NO: 110 - protein, 9 aa, Artificial Sequence
  Description of Artificial Sequence Synthetic peptide
GSDFDCELR

SEQ ID NO: 111 - protein, 9 aa, Artificial Sequence
  Description of Artificial Sequence Synthetic peptide
NTDEMVELR

SEQ ID NO: 112 - protein, 10 aa, Artificial Sequence
  Description of Artificial Sequence Synthetic peptide
GGDLMAYDRR

SEQ ID NO: 113 - protein, 10 aa, Artificial Sequence
  Description of Artificial Sequence Synthetic peptide
GGDLMAYDRR

SEQ ID NO: 114 - protein, 11 aa, Artificial Sequence
  Description of Artificial Sequence Synthetic peptide
NLPLPPPPPPR

SEQ ID NO: 115 - protein, 11 aa, Artificial Sequence
  Description of Artificial Sequence Synthetic peptide
NLPLPPPPPPR

SEQ ID NO: 116 - protein, 11 aa, Artificial Sequence
  Description of Artificial Sequence Synthetic peptide
NLPLPPPPPPR

SEQ ID NO: 117 - protein, 11 aa, Artificial Sequence
  Description of Artificial Sequence Synthetic peptide
IDEPLEGSEDR

SEQ ID NO: 118 - protein, 12 aa, Artificial Sequence
  Description of Artificial Sequence Synthetic peptide
IILDLISESPIK

SEQ ID NO: 119 - protein, 11 aa, Artificial Sequence
  Description of Artificial Sequence Synthetic peptide
SRNTDEMVELR

SEQ ID NO: 120 - protein, 15 aa, Artificial Sequence
  Description of Artificial Sequence Synthetic peptide
LLIHQSLAGGIIGVK

SEQ ID NO: 121 - protein, 12 aa, Artificial Sequence
  Description of Artificial Sequence Synthetic peptide
IIPTLEEYQHYK

SEQ ID NO: 122 - protein, 12 aa, Artificial Sequence
  Description of Artificial Sequence Synthetic peptide
LFQECCPHSTDR

SEQ ID NO: 123 - protein, 14 aa, Artificial Sequence
  Description of Artificial Sequence Synthetic peptide
IILDLISESPIKGR

SEQ ID NO: 124 - protein, 14 aa, Artificial Sequence
  Description of Artificial Sequence Synthetic peptide
RPAEDMEEEQAFKR

SEQ ID NO: 125 - protein, 17 aa, Artificial Sequence
  Description of Artificial Sequence Synthetic peptide
TDYNASVSVPDSSGPER

SEQ ID NO: 126 - protein, 17 aa, Artificial Sequence
  Description of Artificial Sequence Synthetic peptide
TDYNASVSVPDSSGPER

SEQ ID NO: 127 - protein, 17 aa, Artificial Sequence
  Description of Artificial Sequence Synthetic peptide
ALRTDYNASVSVPDSSG

SEQ ID NO: 128 - protein, 460 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDDYDDMSPRRGPPPPPPGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHEGASIKIDEPLEGSEDRIITITGTQDQIQNAQYLLQNSVKQYSGKFF

SEQ ID NO: 129 - protein, 461 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDDYDDMSPRRGPPPPPPGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHEGASIKIDEPLEGSEDRIITITGTQDQIQNAQYLLQNSVKQYADVEGF

SEQ ID NO: 130 - protein, 25 aa, Artificial Sequence
  Description of Artificial Sequence Synthetic peptide
RPAEDMEEEQAFKRSRNTDEMVELR

SEQ ID NO: 131 - protein, 17 aa, Artificial Sequence
  Description of Artificial Sequence Synthetic peptide
ALRTDYNASVSVPDSSG

SEQ ID NO: 132 - protein, 24 aa, Artificial Sequence
  Description of Artificial Sequence Synthetic peptide
GSDFDCELRLLIHQSLAGGIIGVK

SEQ ID NO: 133 - protein, 22 aa, Artificial Sequence
  Description of Artificial Sequence Synthetic peptide
LFQECCPHSTDRVVLIGGKPDR

SEQ ID NO: 134 - protein, 14 aa, Artificial Sequence
  Description of Artificial Sequence Synthetic peptide
IILDLISESPIKGR

SEQ ID NO: 135 - protein, 21 aa, Artificial Sequence
  Description of Artificial Sequence Synthetic peptide
NLPLPPPPPPRGGDLMAYDRR

SEQ ID NO: 136 - protein, 11 aa, Artificial Sequence
  Description of Artificial Sequence Synthetic peptide
IDEPLEGSEDR